(19) United States
(12) Patent Application Publication
(10) Pub. No.: US 2026/0102078 A1
(43) Pub. Date: Apr. 16, 2026

(54) FLUID TRANSFER APPARATUS, SYSTEM AND METHODS FOR USE

(71) Applicant: ResMed Pty Ltd, Bella Vista (AU)

(72) Inventor: John Paul MCKEOWN, Sydney (AU)

(21) Appl. No.: 19/353,764

(22) Filed: Oct. 9, 2025

(30) Foreign Application Priority Data
Oct. 11, 2024 (AU) 2024903282
Aug. 15, 2025 (AU) 2025903672

(51) Int. Cl. A61B 5/08; A61B 5/00; A61M 16/00; A61M 16/06

(52) U.S. Cl. CPC A61B 5/0826; A61B 5/0059; A61M 16/0003; A61M 16/0633

(57) ABSTRACT

A fluid transfer apparatus includes at least one reservoir for containing at least one product; and at least one fluid delivery system fluidly connectable to the reservoir and comprising an application member having a patient contacting surface. The fluid delivery system is configured such that, in use, the fluid delivery system may receive at least one product from the reservoir and provide at least one product in fluid form to the patient contacting surface of the application member.

### 1 BACKGROUND OF THE TECHNOLOGY

### 1.1 Field of the Technology

The present technology relates to one or more of the screening, diagnosis, monitoring, treatment, prevention and amelioration of respiratory-related disorders. The present technology also relates to medical devices or apparatus, systems and their use.

### 1.2 Description of the Related Art

### 1.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hypoventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem, e.g. see U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload, e.g. see U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hypoventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 1.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 1.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

Non-invasive ventilation (NIV) provides ventilatory support to a patient through the upper airways to assist the patient breathing and/or maintain adequate oxygen levels in the body by doing some or all of the work of breathing. The ventilatory support is provided via a non-invasive patient interface. NIV has been used to treat CSR and respiratory failure, in forms such as OHS, COPD, NMD and Chest Wall disorders. In some forms, the comfort and effectiveness of these therapies may be improved.

Invasive ventilation (IV) provides ventilatory support to patients that are no longer able to effectively breathe themselves and may be provided using a tracheostomy tube or endotracheal tube. In some forms, the comfort and effectiveness of these therapies may be improved.

### 1.2.2.2 Flow Therapies

Not all respiratory therapies aim to deliver a prescribed therapeutic pressure. Some respiratory therapies aim to deliver a prescribed respiratory volume, by delivering an inspiratory flow rate profile over a targeted duration, possibly superimposed on a positive baseline pressure. In other cases, the interface to the patient's airways is ‘open’ (unsealed) and the respiratory therapy may only supplement the patient's own spontaneous breathing with a flow of conditioned or enriched gas. In one example, High Flow therapy (HFT) is the provision of a continuous, heated, humidified flow of air to an entrance to the airway through an unsealed or open patient interface at a “treatment flow rate” that may be held approximately constant throughout the respiratory cycle. The treatment flow rate is nominally set to exceed the patient's peak inspiratory flow rate. HFT has been used to treat OSA, CSR, respiratory failure, COPD, and other respiratory disorders. One mechanism of action is that the high flow rate of air at the airway entrance improves ventilation efficiency by flushing, or washing out, expired CO2 from the patient's anatomical deadspace. Hence, HFT is thus sometimes referred to as a deadspace therapy (DST). Other benefits may include the elevated warmth and humidification (possibly of benefit in secretion management) and the potential for modest elevation of airway pressures. As an alternative to constant flow rate, the treatment flow rate may follow a profile that varies over the respiratory cycle.

Another form of flow therapy is long-term oxygen therapy (LTOT) or supplemental oxygen therapy. Doctors may prescribe a continuous flow of oxygen enriched air at a specified oxygen concentration (from 21%, the oxygen fraction in ambient air, to 100%) at a specified flow rate (e.g., 1 litre per minute (LPM), 2 LPM, 3 LPM, etc.) to be delivered to the patient's airway.

### 1.2.2.3 Supplementary Oxygen

For certain patients, oxygen therapy may be combined with a respiratory pressure therapy or HFT by adding supplementary oxygen to the pressurised flow of air. When oxygen is added to respiratory pressure therapy, this is referred to as RPT with supplementary oxygen. When oxygen is added to HFT, the resulting therapy is referred to as HFT with supplementary oxygen.

### 1.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

### 1.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

Certain masks may cause some patients a feeling of claustrophobia, unease and/or may feel overly obtrusive.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

Consequently, some masks suffer from being obtrusive, aesthetically undesirable, costly, poorly fitting, difficult to use, and/or uncomfortable especially when worn for long or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy, especially if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 1.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

### 1.2.3.1.2 Positioning and Stabilising Structure

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face. Several factors may be considered when comparing different positioning and stabilising techniques. These include: how effective the technique is at maintaining the seal-forming structure in the desired position and in sealed engagement with the face during use of the patient interface; how comfortable the interface is for the patient; whether the patient feels intrusiveness and/or claustrophobia when wearing the patient interface; and aesthetic appeal.

One technique is the use of adhesives, e.g. see US Patent Application Publication No. US 2010/0000534.

Another technique is the use of one or more straps and/or stabilising harnesses. Many such harnesses suffer from being one or more of ill-fitting, bulky, uncomfortable and awkward to use.

### 1.2.3.1.3 Pressurised Air Conduit

In one type of treatment system, a flow of pressurised air is provided to a patient interface through a conduit in an air circuit that fluidly connects to the patient interface at a location that is in front of the patient's face when the patient interface is positioned on the patient's face during use. The conduit may extend from the patient interface forwards away from the patient's face.

### 1.2.3.1.4 Pressurised Air Conduit Used for Positioning/Stabilising the Seal-Forming Structure

Another type of treatment system comprises a patient interface in which a tube that delivers pressurised air to the patient's airways also functions as part of the headgear to position and stabilise the seal-forming portion of the patient interface at the appropriate part of the patient's face. This type of patient interface may be referred to as having “conduit headgear” or “headgear tubing”. Such patient interfaces allow the conduit in the air circuit providing the flow of pressurised air from a respiratory pressure therapy (RPT) device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head.

It is desirable for patient interfaces incorporating headgear tubing to be comfortable for a patient to wear over a prolonged duration when the patient is asleep, form an air-tight and stable seal with the patient's face, while also able to fit a range of patient head shapes and sizes.

### 1.2.3.2 Respiratory Pressure Therapy (RPT) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 1.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 1.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 1.2.3.5 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 1.2.3.6 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

### 1.2.3.7 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiography (ECG), electrooculograpy (EOG), electromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information. Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

Traditional diagnostic methods for OSA, such as polysomnography as described above, are often cumbersome, expensive, and require overnight monitoring in a sleep lab. This has led to a growing interest in developing more accessible and cost-effective diagnostic tools.

Photoplethysmography (PPG) is a non-invasive optical technique used to detect blood volume changes in the microvascular bed of tissue. It is commonly used in wearable devices to monitor heart rate and other physiological parameters. However, the effectiveness of PPG can be limited by the opacity of the skin, which can interfere with the accuracy of the measurements. Enhancing skin transparency could significantly improve the performance of PPG-based diagnostics, making it a valuable tool for screening and monitoring conditions like OSA. The development of new methods and apparatus that may be used in conjunction with, or incorporated as part of a new or existing respiratory therapy apparatus may improve the ability to monitor, diagnose and treat respiratory diseases in patients more efficiently, accurately and cost effectively.

Additionally, the integration of machine learning and artificial intelligence (AI) into diagnostic systems holds the promise of more accurate and personalized treatment plans, further advancing the field of medical diagnostics.

### 2 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

One form of the present technology comprises a positioning and stabilising structure configured to provide a force to hold a seal-forming structure in a therapeutically effective position on the patient's head. The positioning and stabilising structure includes at least one strap.

One form of the present technology comprises a patient interface comprising a plenum chamber, a seal-forming structure, and a positioning and stabilising structure.

One form of the present technology comprises patient interface comprising a plenum chamber pressurisable to a therapeutic pressure of at least 4 cmH2O above ambient air pressure. The plenum chamber includes at least one plenum chamber inlet port sized and structured to receive a flow of air at the therapeutic pressure for breathing by a patient. The patient interface also comprises a seal-forming structure that is constructed and arranged to form a seal with a region of the patient's face surrounding an entrance to the patient's airways. The seal-forming structure has a hole therein such that the flow of air at said therapeutic pressure is delivered to at least an entrance to the patient's nares. The seal-forming structure is constructed and arranged to maintain said therapeutic pressure in the plenum chamber throughout the patient's respiratory cycle in use. The patient interface also comprises a positioning and stabilising structure to provide a force to hold the seal-forming structure in a therapeutically effective position on the patient's head.

Another aspect of one form of the present technology is a series of modular elements that may be interconnected in order to form different styles of patient interfaces.

In one form, there are at least two versions or styles of each modular element. The versions or styles may be interchangeably used with one another in order to form different modular assemblies.

One form of the present technology comprises a fluid transfer apparatus.

One form of the present technology comprises a fluid transfer apparatus for use with a RPT system or component thereof.

In some forms of the technology, the fluid transfer apparatus forms part of a patient interface in an RPT system and/or is configured to be removably attached to the patient interface.

In some forms, the fluid transfer apparatus is configured with the patient interface to facilitate connection of the fluid transfer apparatus to the skin of a patient when the patient interface is in use.

In some forms of the technology, the fluid transfer apparatus is configured for attachment to the patient interface at a position to facilitate connection of the fluid transfer apparatus to one or more temples of a patient when the patient interface is in use.

In some forms of the technology, the fluid transfer apparatus is configured for attachment to the patient interface at a position to facilitate connection of the fluid transfer apparatus to the cheek or cheeks of a patient when the patient interface is in use.

In some forms of the technology, the fluid transfer apparatus is configured for attachment to the patient interface at a position to facilitate connection of the fluid transfer apparatus to the forehead of a patient when the patient interface is in use.

In some forms of the technology, the patient interface comprises a forehead support, and the fluid transfer apparatus is configured to form part of the forehead support, and/or configured to be removably attached to the forehead support.

In some forms of the technology, the fluid transfer apparatus forms part of the positioning and stabilising device, and/or is configured to be removably attached to the positioning and stabilising device.

In some forms of the technology, the fluid transfer apparatus may be configured as an over-ear module, optionally connected to the patient interface.

In some forms of the technology, the fluid transfer apparatus may be configured as a finger mounted module.

Another aspect of one form of the present technology is a fluid transfer apparatus, the fluid transfer apparatus comprising:
at least one reservoir for containing a product; and
at least one fluid delivery system fluidly connectable to the reservoir and comprising an application member having a patient contacting surface;
   the fluid delivery system configured such that in use, the fluid delivery system may receive a product from the reservoir and provide that product in fluid form to the patient contacting surface of the application member.

In some forms, the fluid transfer apparatus is configured for use with an RPT system.

In some forms of the technology, the fluid is a dye and/or contrast media. In some forms, the dye is a diagnostic dye.

In some forms, the diagnostic dye is suitable for increasing skin and/or soft tissue transparency.

In some forms, the diagnostic dye is tartrazine.

In some forms of the technology the reservoir is a fluid reservoir in the form of a chamber.

In other forms of the technology, the reservoir is configured to be able to contain a dye powder.

In some forms of the technology, the fluid transfer apparatus comprises two reservoirs.

In some forms, the fluid transfer apparatus is configured to enable mixing of products from the two reservoirs prior to providing the product to the patient contacting surface of the application member.

In some forms, the fluid transfer apparatus further comprises one or more of:
an optical sensor, for example a sensor configured for pulse-oximetry, near-infrared spectroscopy, diffuse-optical tomography, and/or photoacoustic imaging;
a light source, for example an LED or fluorescent tracer;
a transmitter,
a processor or microprocessor;
a storage medium; and
a power source.

In some forms, the fluid transfer apparatus may be configured for use with or prior to using existing sensing devices as part of respiratory therapy.

In some forms, the fluid delivery system comprises and absorbent pad. In some forms the absorbent pad comprises multiple layers of material, or material with regions/patterns of materials having different properties. In some forms the material may have different properties selected from absorbency, moisture wicking, water or fluid resistance, wettability, roughness gradients, patterns, hydrophilicity and hydrophobicity.

In some forms, the application member of the fluid transfer apparatus comprises at least one reservoir interface and a patient contacting surface.

In some forms the at least one reservoir interface is configured to enable the fluid connection with the reservoir to be open, partially open or closed.

Another aspect of the technology is a patient interface for use with a respiratory therapy system, the patient interface comprising a fluid transfer apparatus configured with the patient interface to facilitate connection of the fluid transfer apparatus to the skin of a patient when the patient interface is in use.

An aspect of one form of the present technology is a method of manufacturing apparatus.

According to further forms of the technology, there is provided a method for diagnosing and screening obstructive sleep apnea (OSA). The method involves administering a diagnostic dye to a subject's skin tissue to increase skin transparency, applying and analyzing optical signals through the patient's skin using an optical sensor to diagnose or monitor OSA.

In some forms, the method comprises;
a) administering a diagnostic dye to the subject's skin to increase skin transparency at a measurement site using the fluid transfer apparatus as described above;
b) providing light to the subject's skin tissue at the measurement site using a light source;
c) receiving reflected or transmitted light from the measurement site using an optical sensor;
d) analysing the reflected or transmitted light received from the measurement site to determine at least one measured parameter;
e) comparing the measured parameter to at least one predetermined threshold or at least one range of parameters associated with an indication of OSA;
f) determining if the at least one measured parameter indicates the patient is positive for OSA or negative for OSA, based on a comparison of the at least one measured parameter with the at least one predetermined parameter; and
g) providing a positive or negative diagnosis of OSA based on the determination of step f).

In some forms, the method further comprises repeating at least steps b)-d) multiple times over a period of time and identifying any change in the at least one measured parameter over a specified time period.

In some forms, the light source is a single or multi-frequency LED and the optical sensor is a photodetector.

In some forms, the at least one parameter measured is a change in blood volume in microvascular tissue.

In some forms, the method comprises measuring changes in blood volume over a period of time using photoplethysmography (PPG).

In some forms, the method comprises further comprises using one or more measurement devices in conjunction with the method steps. In some forms, the method comprises measuring blood oxygenation levels using pulse oximetry and a pulse oximetry device.

In some forms, the at least one measured parameter is analyzed to define the phenotypic cause of the subject's OSA.

In some forms of the technology, at least step a) performed using a fluid transfer apparatus as described in further detail above. In some forms the fluid transfer apparatus forms part of a patient interface in an RPT system and/or is configured to be removably attached to the patient interface.

In some forms, one or more of the method steps may be performed by, or in conjunction with the fluid transfer apparatus.

In a further forms of the technology there is provided an OSA diagnostic and screening system is used to accurately diagnose or screen for OSA by analyzing optical signals as they pass through or are reflected from the subject's cardiovascular system and/or tissue.

In some forms, the system comprises a processor to analyze the optical signals in the subject's cardiovascular system and/or tissue to define the phenotypic causes of OSA.

In some forms, the system comprises;
a means to enable increased skin transparency at a measurement site located on a subject;
a light source configured to provide light to the subject's skin tissue at the measurement site;
an optical sensor configured to receive reflected or transmitted light from the measurement site;
a transmitter configured to transmit information from the optical sensor to a processor;
a processor configured to analyse the reflected or transmitted light received by the optical sensor from the measurement site, determine at least one measured parameter and compare the at least one measured parameter to at least one predetermined threshold or at least one range of parameters associated with an indication of OSA to provide an OSA diagnosis; and
an interface configured to provide information about the OSA diagnosis to a user or external device.

In some forms the system further comprises a power source and/or a storage medium.

In some forms, at least the means for administering a diagnostic dye is a fluid transfer device as described above.

In some forms, the optical sensor is configured for pulse-oximetry, near-infrared spectroscopy, diffuse-optical tomography, and/or photoacoustic imaging.

In some forms, the optical signal transmitter is a single or multi-frequency LED and the optical sensor is a photodetector.

In some forms, the system further comprises a means for treating obstructive sleep apnea. In some forms, the means for treating obstructive sleep apnea is selected from at least one of positive airway pressure (PAP) respiratory therapy system or a mandibular adjustment device.

In accordance with other embodiments, a method for treating obstructive sleep apnea (OSA) in a subject is provided, the method comprising;
a) administering a diagnostic dye to a subject's skin tissue to increase skin transparency at a measurement site using a fluid transfer apparatus as described in further detail above;
b) providing light to the subject's skin tissue at the measurement site from a light source;
c) receiving reflected or transmitted light from the measurement site using an optical sensor;
d) analysing the reflected or transmitted light received from the measurement site to determine at least one measured parameter using a processor;
e) comparing the at least one measured parameter to at least one predetermined threshold or at least one range of parameters associated with an indication of OSA;
f) determining if the at least one measured parameter indicates OSA based on comparison with the at least one predetermined parameter;
g) providing a diagnosis of OSA based on a positive or negative determination of OSA; and
h) if a positive diagnosis is determined at step g), providing the subject with treatment of OSA using known OSA treatment methods.

In some forms, the method further comprises repeating at least steps b)-d) multiple times over a period of time and identifying any change in measured parameters over a specified time period.

In some forms, if a positive diagnosis is determined at step g), the method further comprises the step of identifying or suggesting a phenotypic cause of OSA and subsequently recommending an OSA treatment method based on the identification of a phenotypic cause of the OSA.

In accordance with additional embodiments, the known OSA treatment methods comprise at least one of positive airway pressure (PAP) respiratory therapy system, mandibular adjustment devices, or pharmacological treatments.

In some forms, the light source is single or multi-frequency LED and the optical signal detector is a photodetector.

In some forms, the parameter measured is a change in blood volume in microvascular tissue.

In some forms, the method comprises measuring changes in blood volume over a period of time using photoplethysmography (PPG).

In some forms, the method comprises further comprises using one or more measurement devices in conjunction with the method steps. In some forms, the method comprises measuring blood oxygenation levels using pulse oximetry and a pulse oximetry device.

In some forms, optical signals in the subject's cardiovascular system and/or tissue are analyzed to define the phenotypic causes of OSA.

In accordance with further embodiments, the phenotypic causes include one or more of passive critical closing pressure of the upper airway [Pcrit], nonanatomic factors including genioglossus muscle responsiveness, arousal threshold, and respiratory control stability.

In some forms the fluid transfer apparatus forms part of a patient interface in an RPT system and/or is configured to be removably attached to the patient interface.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 3 BRIEF DESCRIPTION OF THE DRAWINGS

The present technology is illustrated by way of example, and not by way of limitation, in the figures of the accompanying drawings, in which like reference numerals refer to similar elements including:

### 3.1 Respiratory Therapy Systems

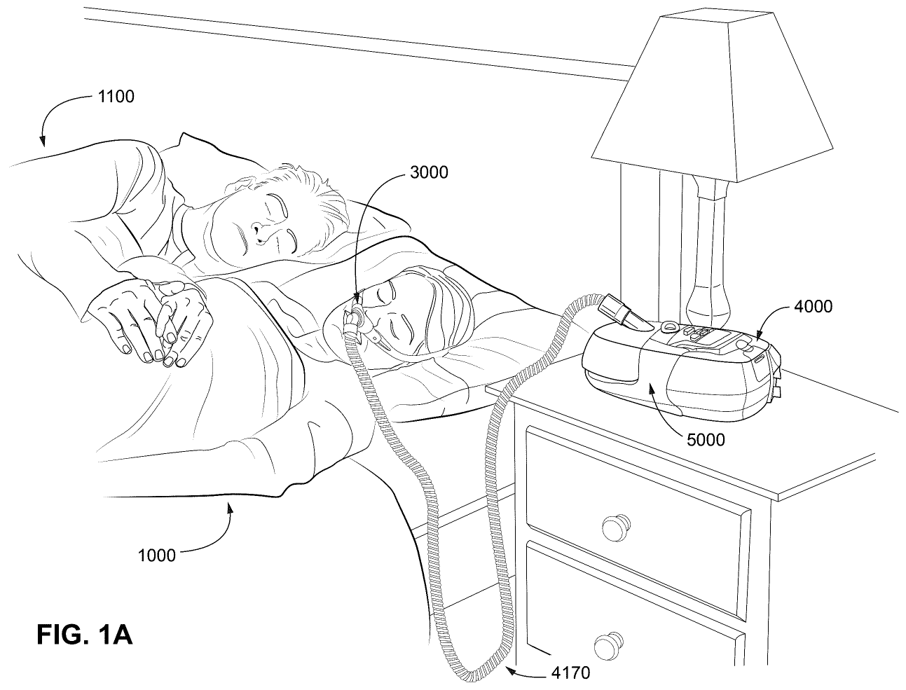

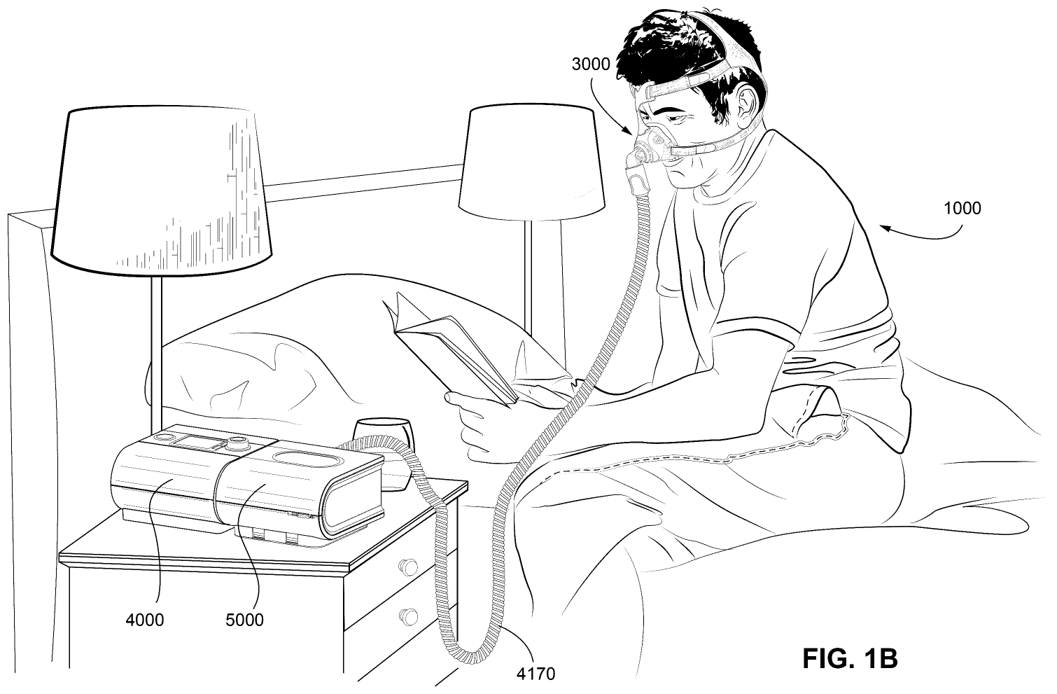

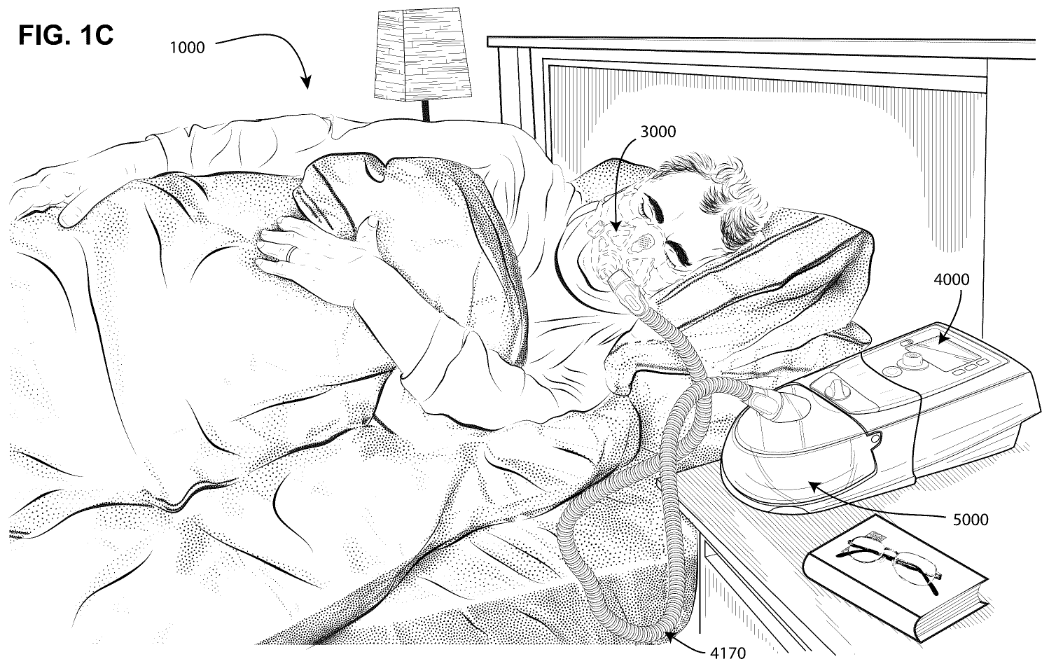

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 3.2 Respiratory System and Facial Anatomy

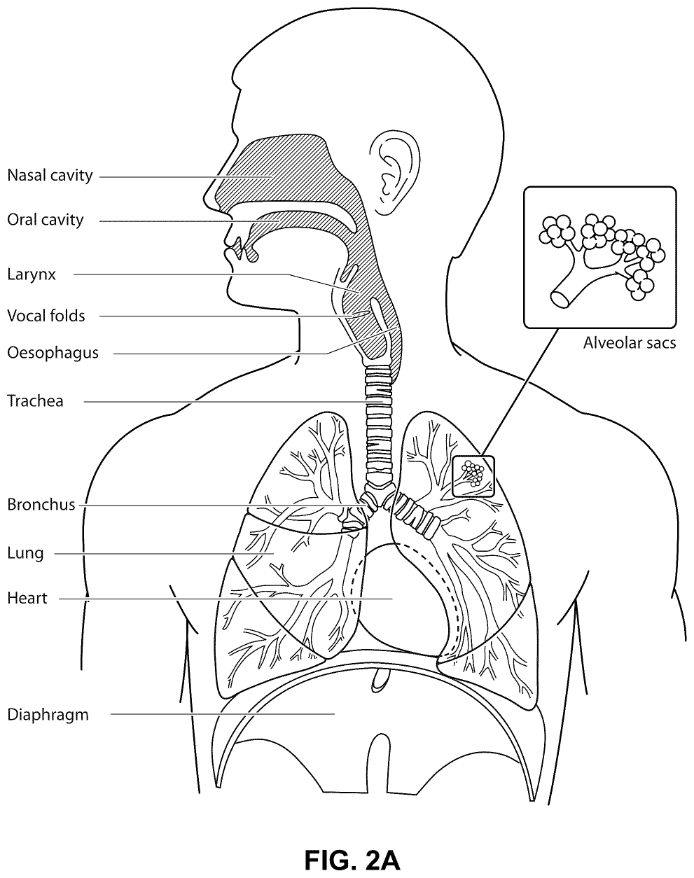

FIG. 2A shows an overview of a human respiratory system including the nasal and oral cavities, the larynx, vocal folds, oesophagus, trachea, bronchus, lung, alveolar sacs, heart and diaphragm.

### 3.3 Patient Interface

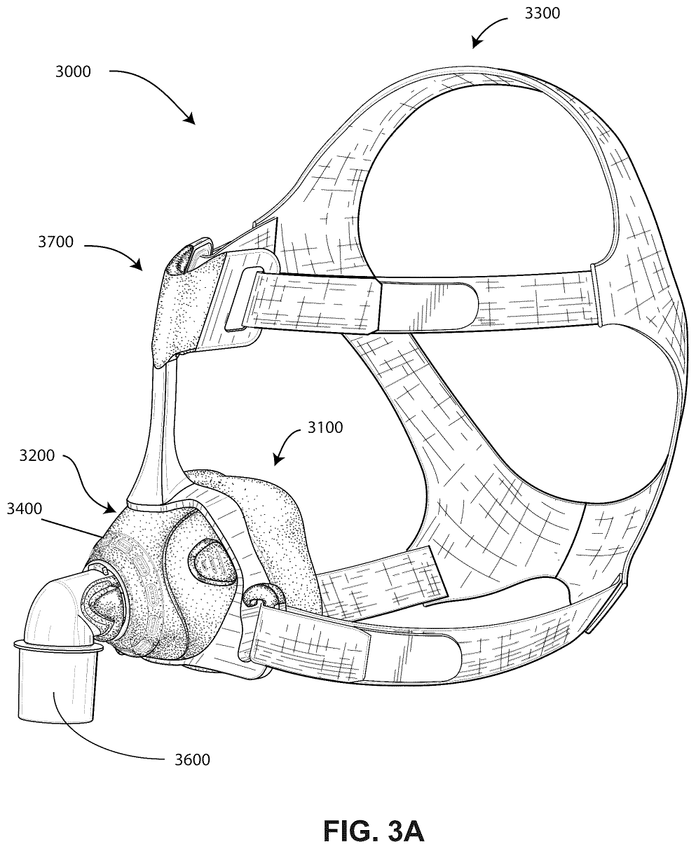

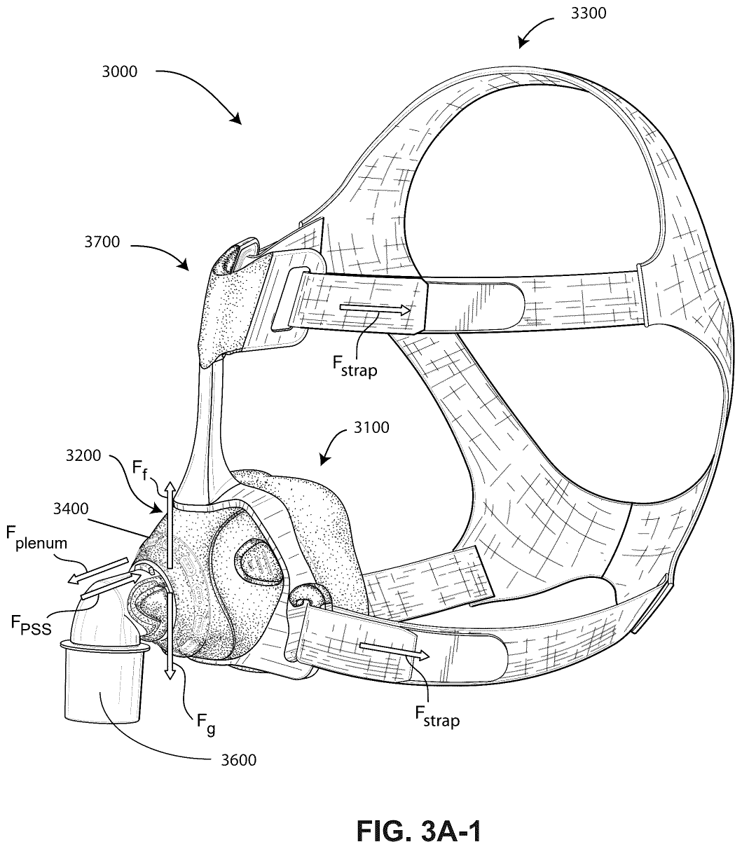

FIG. 3A shows a patient interface in the form of a nasal mask in accordance with one form of the present technology.

FIG. 3A-1 shows forces acting on the patient interface of FIG. 3A, while in use.

FIG. 3B shows a patient interface having conduit headgear, in accordance with one form of the present technology.

### RPT Device

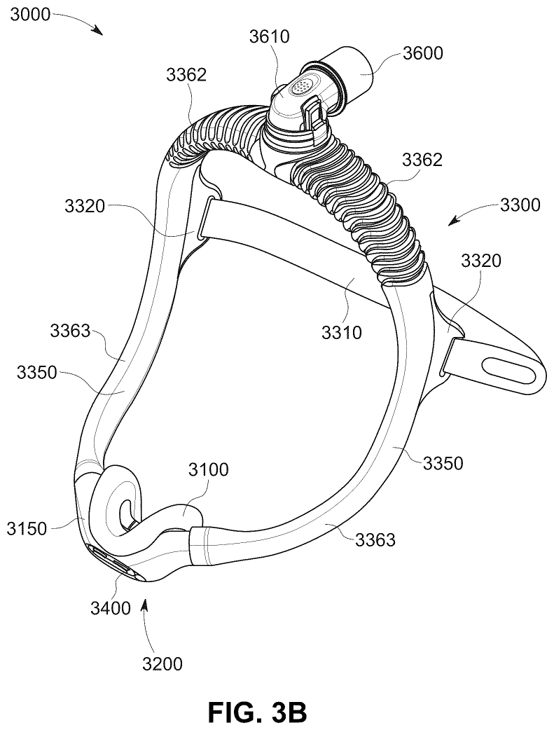

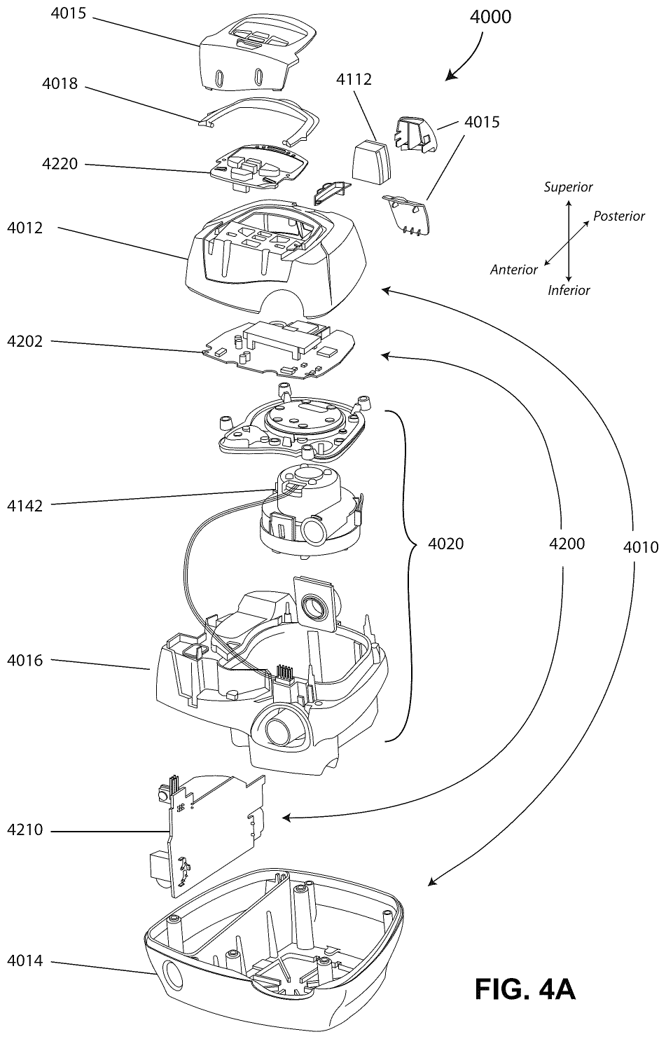

FIG. 4A shows an RPT device in accordance with one form of the present technology.

### Modularity

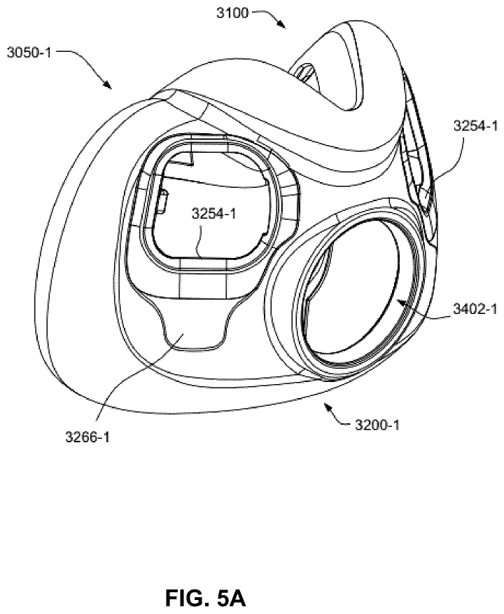

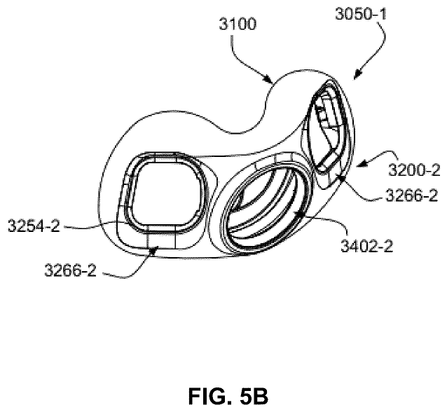

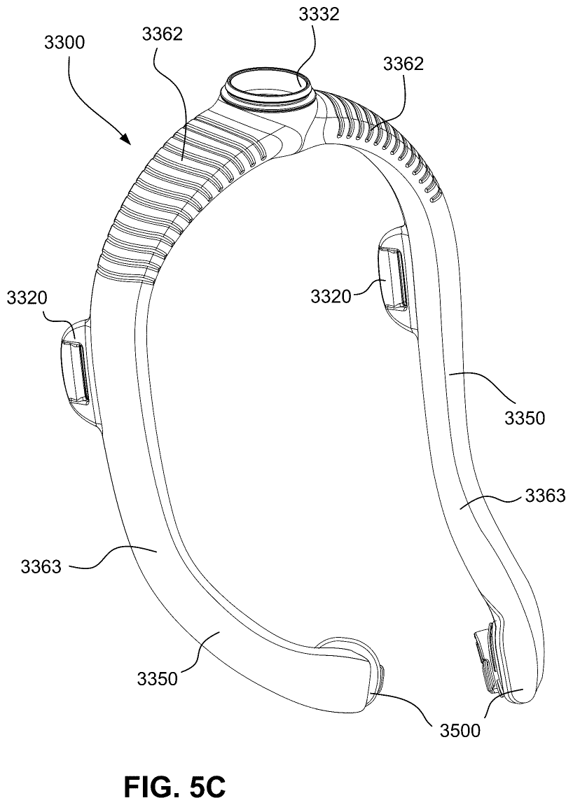

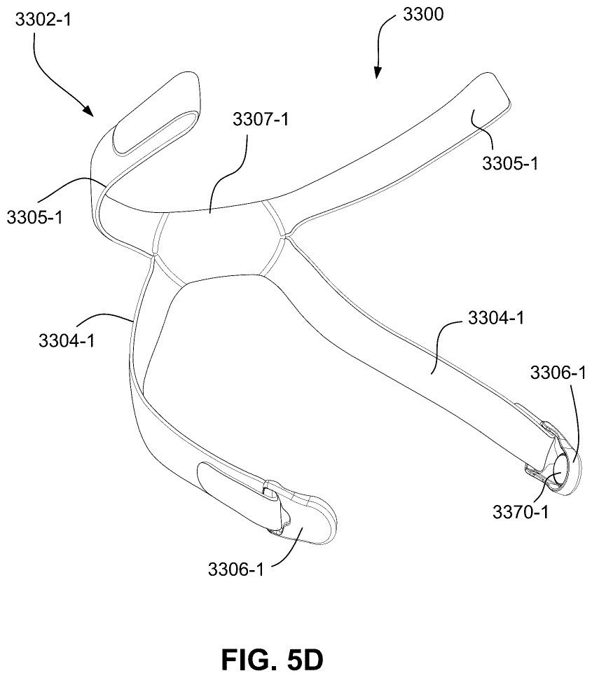

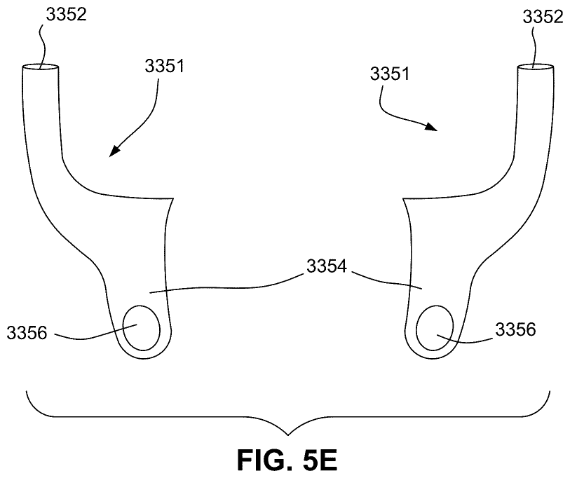

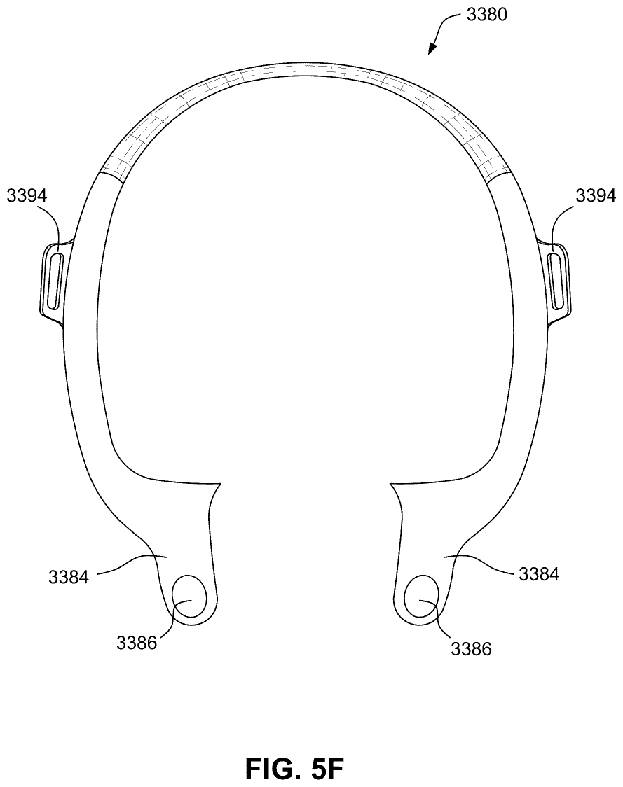

FIG. 5A shows a perspective view of a cushion of a patient interface configured to be worn by a patient and convey pressurized air to the patient's nose and the patient's mouth.

FIG. 5B shows a perspective view of a cushion of a patient interface configured to be worn by a patient and convey pressurized air to the patient's nose.

FIG. 5C shows a perspective view of tubes usable with either the cushion of FIG. 5A or the cushion of FIG. 5B.

FIG. 5D shows a perspective view of headgear straps with rigidiser arms usable with the cushion of FIG. 5A.

FIG. 5E shows a front view of a pair of sleeves that is removably fitted to either the tubes of FIG. 5C or the rigidiser arms of FIG. 5D.

FIG. 5F shows a front view of a full sleeve that is removably fitted to the rigidiser arms of FIG. 5D.

### Fluid Transfer Apparatus

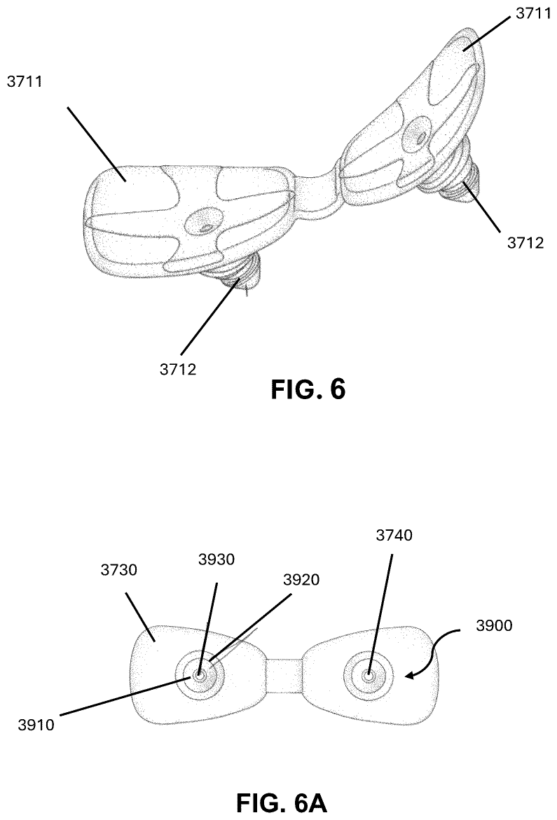

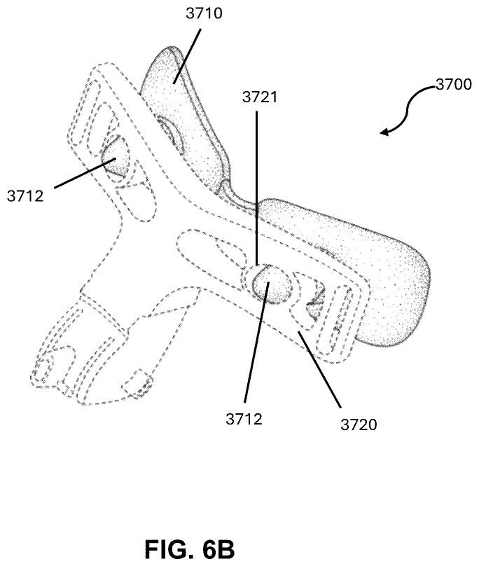

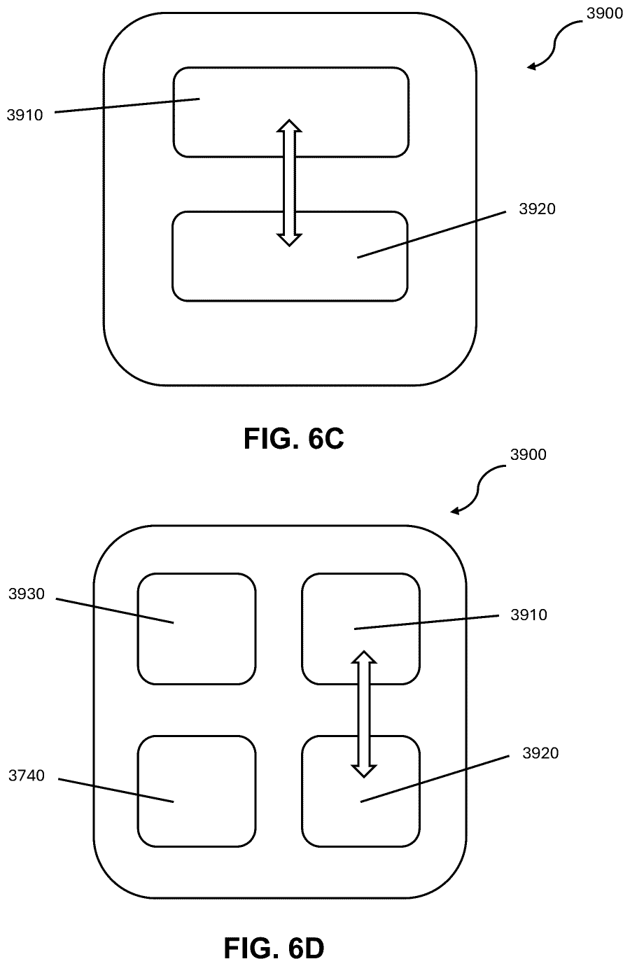

FIG. 6 shows a forehead support for use with the patient interface of FIG. 3A.

FIG. 6A shows a fluid transfer apparatus for use with the forehead support of FIG. 6.

FIG. 6B shows the forehead support of FIG. 6 connected to a support structure of the patient interface.

FIG. 6C shows a simplified diagram of a fluid transfer apparatus in accordance with one form of the present technology.

FIG. 6D shows a simplified diagram of a fluid transfer apparatus in accordance with a further form of the present technology.

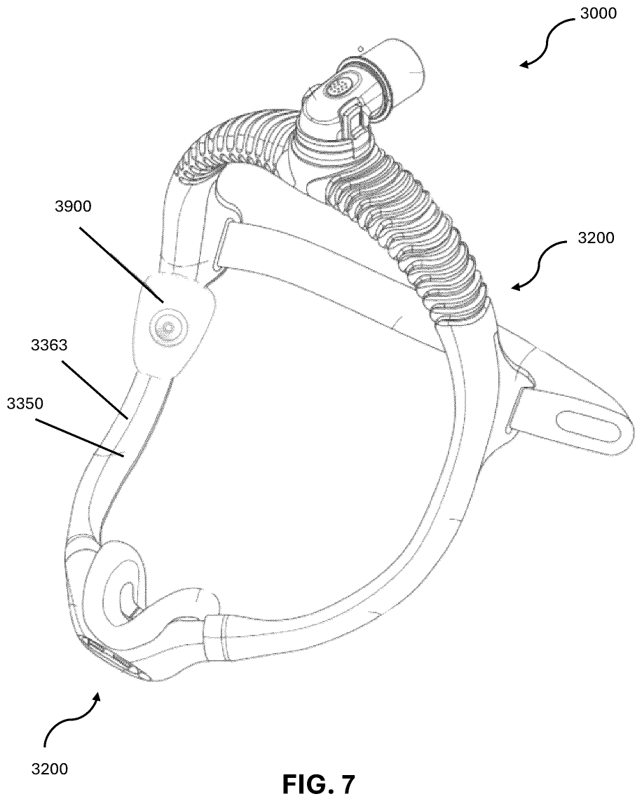

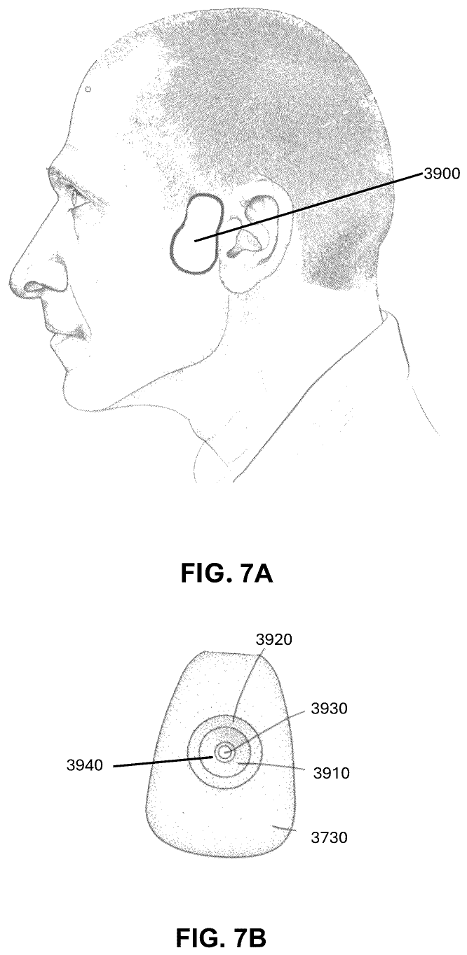

FIG. 7 shows the patient interface of FIG. 3B having conduit headgear, the conduit headgear including a fluid transfer apparatus.

FIG. 7A shows a fluid transfer apparatus at the temple region of a patient's face.

FIG. 7B shows a fluid transfer apparatus for use with the patient interface of FIG. 7.

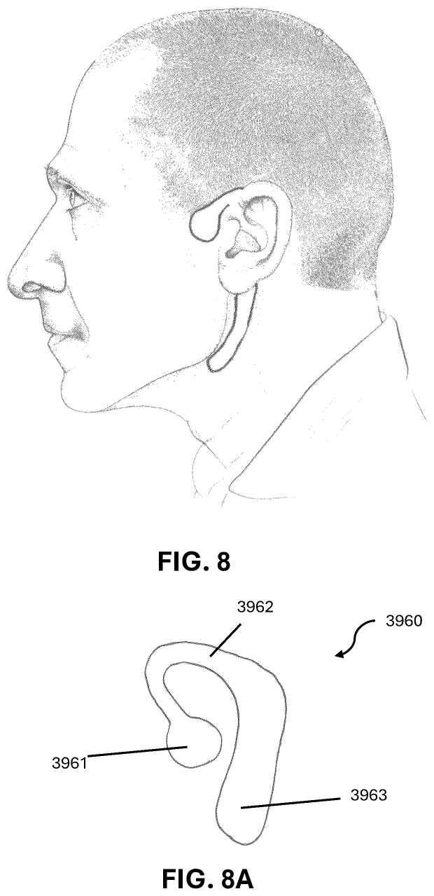

FIG. 8 shows a fluid transfer apparatus in the form of an over-ear device being worn by a patient.

FIG. 8A shows am over-ear embodiment of the fluid transfer apparatus in one form of the technology.

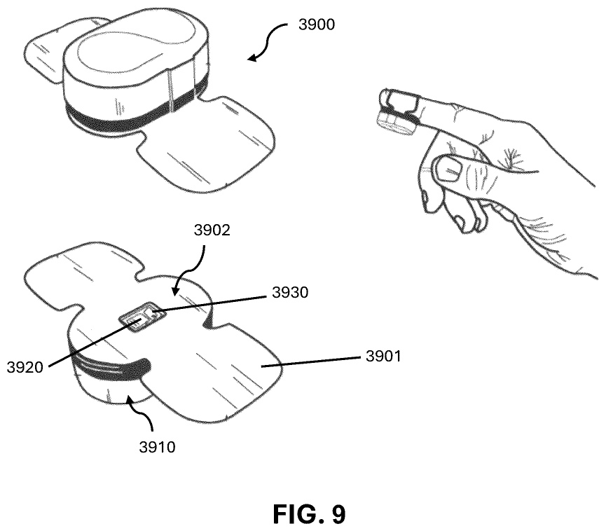

FIG. 9 shows a finger-worn embodiment of the fluid transfer apparatus in one form of the technology.

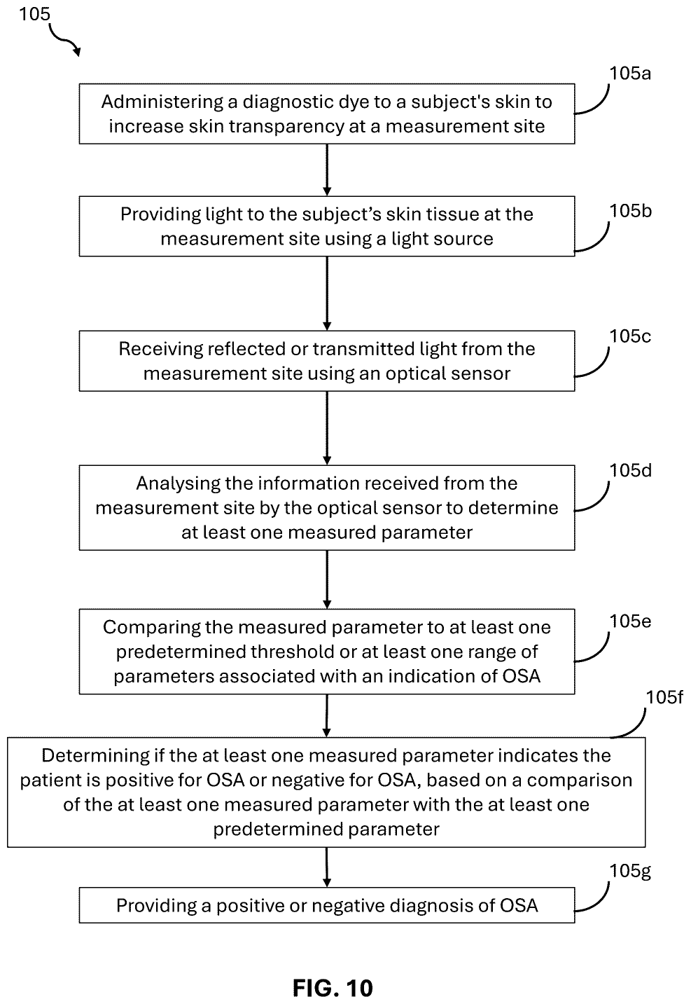

FIG. 10 shows a method for the diagnosis or screening of OSA in one form of the technology.

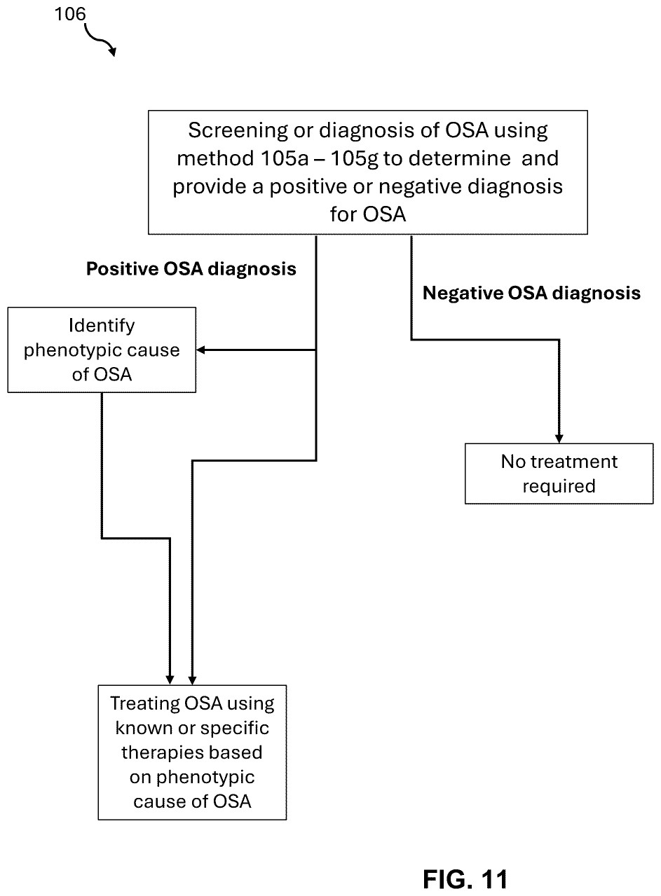

FIG. 11 shows a method for the treatment of a subject with OSA following a positive or negative diagnosis in one form of the technology

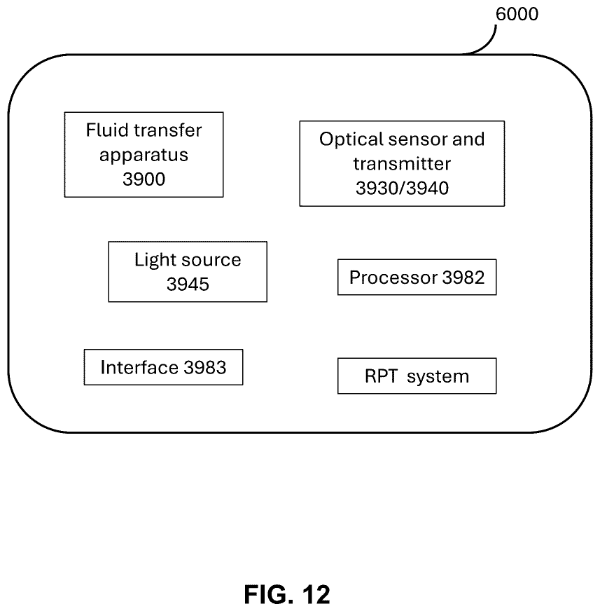

FIG. 12 shows a diagnostic system for use in the diagnosis, screening and optional treatment of OSA.

### 4 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 4.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 4.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 4.3 Patient Interface

A non-invasive patient interface 3000, such as that shown in FIG. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

As shown in FIG. 3B, a non-invasive patient interface 3000 in accordance with another aspect of the present technology comprises the following functional aspects: a seal-forming structure 3100, a plenum chamber 3200, a positioning and stabilising structure 3300, a vent 3400 and one form of connection port 3600 for connection to an air circuit (such as the air circuit 4170 shown in FIGS. 1A-IC). The plenum chamber 3200 may be formed of one or more modular components (e.g., a cushion module 3150 together with the seal-forming structure 3100) in the sense that it or they can be replaced with different components, for example components of a different size.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure above the ambient, for example at least 2, 4, 6, 10, or 20 cmH2O with respect to ambient.

### 4.3.1 Seal-Forming Structure

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs—the actual sealing surface—may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head.

### 4.3.1.1 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 4.3.1.2 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 4.3.1.3 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 4.3.1.4 Forehead Region

In one form, the seal-forming structure that forms a seal in use on a forehead region of the patient's face. In such a form, the plenum chamber may cover the eyes in use.

### 4.3.1.5 Nose-Only Masks

In one form, the patient interface 3000 comprises a seal-forming structure 3100 configured to seal around an entrance to the patient's nasal airways but not around the patient's mouth. The seal-forming structure 3100 may be configured to seal to the patient's lip superior. The patient interface 3000 may leave the patient's mouth uncovered. This patient interface 3000 may deliver a supply of air or breathable gas to both nares of patient 1000 and not to the mouth. This type of patient interface may be identified as a nose-only mask.

One form of nose-only mask according to the present technology is what has traditionally been identified as a “nasal mask”, having a seal-forming structure 3100 configured to seal on the patient's face around the nose and over the bridge of the nose. A nasal mask may be generally triangular in shape. In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 1B has this type of seal-forming structure 3100. This patient interface 3000 may deliver a supply of air or breathable gas to both nares of patient 1000 through a single orifice.

Another form of nose-only mask may seal around an inferior periphery of the patient's nose without engaging the user's nasal ridge. This type of patient interface 3000 may be identified as a “nasal cradle” mask and the seal-forming structure 3100 may be identified as a “nasal cradle cushion”, for example. In one form, for example as shown in FIG. 3B, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of a pronasale region of the patient's nose and to the patient's nasal alae. The seal-forming structure 3100 may seal to the patient's lip superior. The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's nares.

In some forms, a nose-only mask may comprise nasal pillows, described above.

### 4.3.1.6 Nose and Mouth Masks

In one form, the patient interface 3000 comprises a seal-forming structure 3100 configured to seal around an entrance to the patient's nasal airways and also around the patient's mouth. The seal-forming structure 3100 may be configured to seal to the patient's face proximate a chin region. This patient interface 3000 may deliver a supply of air or breathable gas to both nares and to the mouth of patient 1000. This type of patient interface may be identified as a nose and mouth mask.

One form of nose-and-mouth mask according to the present technology is what has traditionally been identified as a “full-face mask”, having a seal-forming structure 3100 configured to seal on the patient's face around the nose, below the mouth and over the bridge of the nose. A nose-and-mouth mask may be generally triangular in shape. In one form the patient interface 3000 comprises a seal-forming structure 3100 that forms a seal in use to a patient's chin-region (which may include the patient's lip inferior and/or a region directly inferior to the lip inferior), to the patient's nose bridge or at least a portion of the nose ridge superior to the pronasale, and to cheek regions of the patient's face. The patient interface 3000 shown in FIG. 1C is of this type. This patient interface 3000 may deliver a supply of air or breathable gas to both nares and mouth of patient 1000 through a single orifice. This type of seal-forming structure 3100 may be referred to as a “nose-and-mouth cushion”.

In another form the patient interface 3000 comprises a seal-forming structure 3100 that forms a seal in use on a patient's chin region (which may include the patient's lip inferior and/or a region directly inferior to the lip inferior), to an inferior and/or an anterior surface of a pronasale portion of the patient's nose, to the alae of the patient's nose and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 3000 may have a nasal portion and an oral portion, the nasal portion sealing to the patient's face at similar locations to a nasal cradle mask.

In a further form of nose and mouth mask, the patient interface 3000 may comprise a seal-forming structure 3100 having a nasal portion comprising nasal pillows and an oral portion configured to form a seal to the patient's face around the patient's mouth.

In some forms, the seal-forming structure 3100 may have a nasal portion that is separate and distinct from an oral portion. In other forms, a seal-forming structure 3100 may form a contiguous seal around the patient's nose and mouth.

It is to be understood that the above examples of different forms of patient interface 3000 do not constitute an exhaustive list of possible configurations. In some forms a patient interface 3000 may comprise a combination of different features of the above described examples of nose-only and nose and mouth masks.

### 4.3.1.7 Plenum Chamber

The plenum chamber 3200 has a perimeter that is shaped to be complementary to the surface contour of the face of an average person in the region where a seal will form in use. In use, a marginal edge of the plenum chamber 3200 is positioned in close proximity to an adjacent surface of the face. Actual contact with the face is provided by the seal-forming structure 3100. The seal-forming structure 3100 may extend in use about the entire perimeter of the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is constructed from a translucent material. The use of a translucent material can reduce the obtrusiveness of the patient interface, and help improve compliance with therapy.

In some forms, the plenum chamber 3200 is constructed from a rigid material such as polycarbonate. The rigid material may provide support to the seal-forming structure.

In some forms, the plenum chamber 3200 is constructed from a flexible material (e.g., constructed from a soft, flexible, resilient material like silicone, textile, foam, etc.). For example, in examples then may be formed from a material which has a Young's modulus of 0.4 GPa or lower, for example foam. In some forms of the technology the plenum chamber 3200 may be made from a material having Young's modulus of 0.1 GPa or lower, for example rubber. In other forms of the technology the plenum chamber 3200 may be made from a material having a Young's modulus of 0.7 MPa or less, for example between 0.7 MPa and 0.3 MPa. An example of such a material is silicone.

### 4.3.1.8 Multiple Openings

As shown in FIGS. 5A and 5B, different plenum chambers 3200-1, 3200-2 may be formed as part of a multi-opening cushion 3050-1, 3050-2. In the illustrated examples, the cushions 3050-1, 3050-2 each include three openings, although an alternate cushion may be formed with greater or fewer openings.

In some forms, the different openings may serve different functions. For example, some openings may be exclusively inlet openings, while other openings may be exclusively outlet openings.

In other forms, at least one opening may serve two different functions. For example, one opening may operate as both an inlet and an outlet during the same breathing cycle.

The plurality of openings may allow for a variety of configurations of air delivery to the plenum chamber 3200-1, 3200-2. For example, depending on patient need and/or patient comfort, the patient may use a given cushion 3050-1, 3050-2 in a “tube-up” configuration (e.g., using conduit headgear—described below) or a “tube-down” configuration (e.g., using a single conduit in front of the patient's face).

### 4.3.1.8.1 Nose and Mouth Mask

As shown in FIG. 5A, the plenum chamber 3200-1 includes a pair of plenum chamber inlet ports 3254-1, which may be used to convey gas into and/or out of the plenum chamber 3200-1. The plenum chamber inlet ports 3254-1 may be disposed on opposite sides (e.g., left and right sides) of the plenum chamber 3200-1.

In some forms, the plenum chamber 3200-1 may also include at least one vent opening 3402-1 (see e.g., FIG. 5A). The vent opening 3402-1 may be disposed in a center of the plenum chamber 3200-1. For example, the vent opening 3402-1 may be disposed between the plenum chamber inlet ports 3254-1.

In some forms, the plenum chamber 3200-1 may include a pair of grooves 3266-1. Each groove 3266-1 may be disposed proximate to one of the plenum chamber inlet ports 3254-1. Each groove 3266-1 may form a partially recessed surface.

### 4.3.1.8.2 Nose-Only Mask

The plenum chamber 3200-2 of a nasal only cushion 3050-2 may be similar to the plenum chamber 3200-1 of the mouth and nose cushion 3050-1. Only some similarities and differences between the plenum chambers 3200-1, 3200-2 may be described below.

As shown in FIG. 5B, the plenum chamber 3200-2 includes a pair of plenum chamber inlet ports 3254-2, which may be used to convey gas into and/or out of the plenum chamber 3200-2. The plenum chamber inlet ports 3254-2 may be disposed on opposite sides (e.g., left and right sides) of the plenum chamber 3200-2.

In some forms, the plenum chamber 3200-2 may also include at least one vent opening 3402-2 (see e.g., FIG. 5B). The vent opening 3402-2 may be disposed in a center of the plenum chamber 3200-2. For example, the vent opening 3402-2 may be disposed between the plenum chamber inlet ports 3254-2.

In some forms, the plenum chamber 3200-2 may include a pair of grooves 3266-2. Each groove 3266-2 may be disposed proximate to one of the plenum chamber inlet ports 3254-2. Each groove 3266-2 may form a partially recessed surface.

### 4.3.2 Positioning and Stabilising Structure

The seal-forming structure 3100 of the patient interface 3000 of the present technology may be held in sealing position in use by the positioning and stabilising structure 3300. The positioning and stabilising structure 3300 may comprise and function as “headgear” since it engages the patient's head in order to hold the patient interface 3000 in a sealing position. Examples of a positioning and stabilising structure may be shown in FIGS. 3A and 3A-1.

In one form the positioning and stabilising structure 3300 provides a retention force at least sufficient to overcome the effect of the positive pressure in the plenum chamber 3200 to lift off the face (i.e., Fplenum).

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap,

In certain forms of the present technology, a system is provided comprising more than one positioning and stabilising structure 3300, each being configured to provide a retaining force to correspond to a different size and/or shape range. For example the system may comprise one form of positioning and stabilising structure 3300 suitable for a large sized head, but not a small sized head, and another. suitable for a small sized head, but not a large sized head.

### 4.3.3 Conduit Headgear

### 4.3.3.1 Conduit Headgear Tubes

In some forms of the present technology, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 3B, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth) in use. This allows the conduit of air circuit 4170 providing the flow of pressurised air to connect to a connection port 3600 of the patient interface in a position other than in front of the patient's face, for example on top of the patient's head.

In the form of the present technology illustrated in FIG. 3B, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3610 on top of the head of the patient 1000. This form of technology may be advantageous because, if a patient sleeps with their head on its side and one of the tubes 3350 is compressed to block or partially block the flow of gas along the tube 3350, the other tube 3350 remains open to supply pressurised gas to the patient. In other examples of the technology, the patient interface 3000 may comprise a different number of tubes, for example one tube, or two or more tubes.

The tubes 3350 may be formed from a flexible material, such as an elastomer, e.g. silicone or TPE, and/or from one or more textile and/or foam materials. The tubes 3350 may have a preformed shape and may be able to be bent or moved into another shape upon application of a force but may return to the original preformed shape in the absence of said force. The tubes 3350 may be generally arcuate or curved in a shape approximating the contours of a patient's head between the top of the head and the nasal or oral region.

Each tube 3350 may be configured to receive a flow of air from the connection port 3600 on top of the patient's head and to deliver the flow of air to the seal-forming structure 3100 at the entrance of the patient's airways. In the example shown in FIG. 3B, each tube 3350 lies in use on a path extending from the plenum chamber 3200 across the patient's cheek region and superior to the patient's ear to the elbow 3610. For example, a portion of each tube 3350 proximate the plenum chamber 3200 may overlie a maxilla region of the patient's head in use. Another portion of each tube 3350 may overlie a region of the patient's head superior to an otobasion superior of the patient's head. Each of the tubes 3350 may also lie over the patient's sphenoid bone and/or temporal bone and either or both of the patient's frontal bone and parietal bone. The elbow 3610 may be located in use over the patient's parietal bone, over the frontal bone and/or over the junction therebetween (e.g. the coronal suture).

### 4.3.3.2 Conduit Headgear Connection Port

In certain forms of the present technology, the patient interface 3000 may comprise a connection port 3600 located proximal to a superior, lateral or posterior portion of a patient's head. For example, in the form of the present technology illustrated in FIG. 3B, the connection port 3600 is located on top of the patient's head (e.g. at a superior location with respect to the patient's head). In this example the patient interface 3000 comprises an elbow 3610 forming the connection port 3600. The elbow 3610 may be configured to fluidly connect with a conduit of an air circuit 4170. The elbow 3610 may be configured to swivel with respect to the positioning and stabilising structure 3300 to at least partially decouple the conduit from the positioning and stabilising structure 3300. In some examples the elbow 3610 may be configured to swivel by rotation about a substantially vertical axis and, in some particular examples, by rotation about two or more axes. In some examples the elbow may comprise or be connected to the tubes 3350 by a ball-and-socket joint. The connection portion 3600 may be located in the sagittal plane of the patient's head in use.

### 4.3.3.3 Headgear Tube Fluid Connections

The two tubes 3350 are fluidly connected at their inferior ends to the plenum chamber 3200. In certain forms of the technology, the connection between the tubes 3350 and the plenum chamber 3200 is achieved by connection of two rigid connectors. The tubes 3350 and plenum chamber 3200 may be configured to enable the patient to easily connect the two components together in a reliable manner.

### 4.3.3.4 Four-Point Connection

As shown in FIG. 5D, some forms of the headgear 3302-1 may be a four-point connection headgear. This means that the headgear 3302-1 may connect to four separate places on the plenum chamber 3200, on a frame connected to the plenum chamber 3200, and/or on arms connected to the plenum chamber 3200. The headgear 3302-1 may include four different straps providing a tensile force to help maintain the seal-forming structure 3100 in a sealing position. The positioning and stabilising structure 3300 of FIG. 3A may also be considered a four-point connection headgear.

In some forms, the headgear 3302-1 may include inferior straps 3304-1, which may connect to an inferior portion of the cushion 3050-1. The inferior straps 3304-1 may extend along the patient's cheek toward a posterior region of the patient's head. For example, the inferior straps 3304-1 may overlay the masseter muscle on either side of the patient's face. The inferior straps 3304-1 may therefore contact the patient's head below the patient's ears. The inferior straps 3304-1 may meet at the posterior of the patient's head, and may overlay the occipital bone and/or the trapezius muscle.

The headgear 3302-1 may also include superior straps 3305-1, which may overlay the temporal bones, parietal bone, and/or occipital bone. The superior straps 3305-1 may also connect to the tubes 3350 (e.g., by interfacing with the tabs 3320).

A rear strap 3307-1 may extend between the superior straps 3305-1 and between the inferior straps 3304-1. The inferior and superior straps 3304-1, 3305-1 on a given side (e.g., left or right) may also be connected to the rear strap 3307-1 adjacent to one another. The height of the rear strap 3307-1 may therefore be approximately the combined height of the inferior and superior strap 3304-1, 3305-1. The rear strap 3307-1 may overlay the occipital bone and/or the pariental bone in use. This may allow the rear strap 3307-1 to assist in anchoring the headgear 3302-1 to the patient's head.

In the illustrated example, the headgear 3302-1 may be formed with a substantially X-shape. The inferior and superior straps 3304-1, 3305-1 may be connected to a rear strap 3307-1 using stitching, ultrasonic welding, or any similar process.

### 4.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

Connection port 3600 allows for connection to the air circuit 4170.

### 4.3.5 Forehead Support

In one form, the patient interface 3000 includes a forehead support 3700 as seen in FIGS. 3A and 3B, 6, 6A and 6B. Forehead support 3700 may comprise a forehead frame 3720, held in position on a user's forehead by a positioning and stabilising structure 3300. Forehead support 3700 may be further directly or indirectly connected to a plenum chamber, which is itself stabilised by positioning and stabilising structure 3300. In order to provide comfort in use, forehead support 3700 may comprise comfort pads 3730, comfort pads 3730 supported by and connected to a forehead frame 3720 via forehead backing plates 3711.

### 4.3.6 Anti-Asphyxia Valve

In one form, the patient interface 3000 includes an anti-asphyxia valve.

### 4.3.7 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

### 4.3.8 Modularity

As described above, the cushion, headgear, and sleeves may come in different styles, which may correspond to different uses (e.g., mouth breathing, nasal breathing, etc.). A patient or clinician may select certain combinations of cushions, headgear, and sleeves in order to optimize the effectiveness of the therapy and/or the individual patient's comfort. An example of this sort of modular design is described in PCT/SG2022/050777 filed 28 Oct. 2022, incorporated herein by reference in its entirety.

In some forms, the different styles of cushions, headgear, and sleeves may be used interchangeably with one another in order to form different combinations of patient interfaces. This may be beneficial from a manufacturing prospective because wider variety of patient interfaces may be created using fewer parts. Additionally or alternatively, the various combinations may allow a patient to change styles of patient interface without changing the every component.

Air may be delivered to the patient in one of two main ways. In one example, the patient may receive the flow of pressurized air through headgear tubes 3350 (see e.g., FIG. 3B). This may be referred to as a “tube up” configuration and may position a connection port at the top of the patient's head. In other example, the patient may receive the flow of pressurized air through a conduit connected to the plenum chamber 3200, for example through the connection port 3600 (see e.g., FIG. 3A). This may be referred to a “tube down” configuration where the airflow conduit is positioned in front of the patient's face. Different patients may be more comfortable with one style of air delivery over the other (e.g., because of the patient's sleep style). Therefore, it may be beneficial to allow a single style of patient interface to be used in either the “tube up” or “tube down” configuration.

The patient interface may be part of a modular assembly with a variety of interchangeable components that may be swapped out by a patient and/or clinician for one or more components for a different style. The following description describes the various combinations that may be created by assembling the different components together.

### 4.3.8.1 Sleeve

In some forms, to allow for modularity, a sleeve may be used with the tubes 3350 and/or the rigidisier arms 3340. The sleeve may at least partially surround the tubes 3350 and/or the rigidiser arms 3340. As shown in FIGS. 5E to 5F, different shapes of sleeves may be used, which may correspond to different types of positioning and stabilising structures 3300. In some forms, the configuration of the sleeve may be customized to fit a particular user's face. For instance, the sleeves may be configured in a relatively more posterior region of the patient's head.

In some forms, the sleeve may be constructed from a comfortable material. For example, the sleeve may be constructed from a textile material, a foam material, or a combination of the two. The comfortable material may contact the patient in use, and may feel soft against the patient's skin in order to improve patient compliance.

The material may also be flexible in order to assist in donning or doffing the sleeve from the tube 3350 or the rigidiser arms 3340. For example, the material may allow the sleeve to bend in order to conform to the shape of the tubes or conduit headgear 3350 or the rigidiser arms 3340, which may change depending on the shape of an individual patient's head.

In some forms, the sleeve may also be at least partially elastic (e.g., the material may allow the sleeve to stretch). The elastic material may help the sleeve stretch in order to fit around the tubes 3350 or the rigidiser arms 3340. The elastic material may then return to an initial position that is snug against the tubes 3350 or the rigidiser arms 3340 in order to limit the sleeve from sliding while in use.

As described in more detail below, some forms of the sleeves may be specific to a rigidising element (e.g., tubes 3350 and/or rigidiser arms 3340). However, the sleeves may assist the rigidising elements in connecting interchangeably with the version or styles of cushions (e.g., the mouth and nose cushion 3050-1, the nose-only cushion 3050-2, etc.).

### 4.3.8.1.1 Conduit Sleeve

As shown in FIG. 5E, one example of a sleeve is a conduit sleeve 3351, which may be usable with the tubes 3350 described above.

As shown in FIG. 5E, the conduit sleeve 3351 may include a curved shape that may be similar to the shape of the tubes 3350 shown in FIG. 5C. The flexible material used to construct the conduit sleeve 3351 may allow the conduit sleeve 3351 to further curve in order to correspond to the shape of the tubes 3350 (e.g., when worn by the patient).

In some forms, the conduit sleeve 3351 may include a first or superior opening 3352. The superior opening 3352 may be disposed at one end of the conduit sleeve 3351. The superior opening 3352 may be an opening to a passage that extends along at least a portion of the conduit sleeve 3351.

As shown in FIG. 5E, some forms of the conduit sleeve 3351 may also include an inferior extension 3354. The inferior extension 3354 may be positioned on an opposite end of the conduit sleeve 3351 from the superior opening 3352. The conduit sleeve 3351 may be customized to fit a particular user's face. For instance, the inferior extension 3354 of the conduit sleeve 3351 may be configured in a relatively more posterior region or anterior region of the patient's head.

Some forms of the inferior extension 3354 may include a rigid or semi-rigid piece (e.g., within the sleeve 3351). The rigid or semi-rigid piece may be constructed from a plastic material, or a similar material. Alternatively, the inferior extension 3354 may be stiffened using a manufacturing process (e.g., stitching rigidised thread, flat knitting, using thicker material).

As shown in FIG. 5E, some forms of the inferior extension 3354 may include a connection member 3356. In the illustrated example, the connection member 3356 may be a magnet, although in other examples, the connection member 3356 may be a different type of connector (e.g., a mechanical fastener, an adhesive, hook and loop material, etc.). The connection member 3356 may also be positioned at an end of the inferior extension 3354, although the connection member 3356 could alternatively be positioned anywhere along the inferior extension 3354.

In some forms, the connection member 3356 (e.g., a magnet) may be removably connected to the magnets 3370-1 of the headgear 3302-1. For example, when the conduit sleeves 3351 are connected to the tubes 3350 (see e.g., FIG. 7J), the magnets 3370-1 connected to the inferior straps 3304-1 may be removably connected to the connection member 3356 in order to provide the tensile force.

### 4.3.8.1.2 Four-Point Arm Sleeve

As shown in FIG. 5F, another example of a sleeve is a four-point arm sleeve 3380, which may be usable with the rigidiser arms 3340 described above.

As shown in FIG. 5F, the four-point arm sleeve 3380 may include a curved shape that may be similar to the shape of the rigidiser arm 3340 shown in FIG. 5D. The flexible material used to construct the four-point arm sleeve 3380 may allow the four-point arm sleeve 3380 to further curve in order to correspond to the shape of the rigidiser arm 3340 (e.g., when worn by the patient and/or went bent by the patient).

As shown in FIG. 5F, some forms of the four-point arm sleeve 3380 may include an inferior extension 3384. The inferior extension 3384 may be positioned at an end of the four-point arm sleeve 3380.

In the illustrated example, the shape and/or structure of the inferior extension 3384 is substantially the same as the shape of the inferior extension 3354. For example, the inferior extension 3384 may be more rigid as compared to the rest of the four-point arm sleeve 3380 (e.g., as a result of rigidising thread or rigid material).

As shown in FIG. 5F, some forms of the inferior extension 3384 may include a connection member 3386. In the illustrated example, the connection member 3386 may be a magnet, although in other examples, the connection member 3386 may be a different type of connector (e.g., a mechanical fastener, an adhesive, hook and loop material, etc.). The connection member 3386 may also be positioned at an end of the inferior extension 3384, although the connection member 3386 could alternatively be positioned anywhere along the inferior extension 3384.

In some forms, the connection member 3386 (e.g., a magnet) may be removably connected to the magnets 3370-1 of the headgear 3302-1. For example, when the four-point arm sleeves 3380 are connected to the rigidiser arm 3340 (see e.g., FIG. 7K), the magnets 3370-1 connected to the inferior straps 3304-1 may be removably connected to the connection member 3386 in order to provide the tensile force.

As shown in FIG. 5F, the four-point arm sleeve 3380 may include a pair of tabs 3394, which may be similar to the tab 3320 on the tubes 3350. When the four-point arm sleeve 3380 is worn by the patient, the tabs 3394 may be positioned in substantially the same place on the patient's head as where the tabs 3320 are positioned when the patient wears the tubes 3350.

### 4.4 Fluid Transfer Apparatus

The use of pulse-oximetry and other known optical imaging techniques to determine the oxygen saturation in blood are often used in the diagnosis and treatment of OSA. By establishing the oxygen levels in the blood, determinations can be made to increase or decrease PRT therapy, supplement an RPT system with oxygen or diagnosis a medical problem.

The application of a fluid in the form of a dye to the patient's skin prior to conducting an optical imaging measurement at the same site has been shown to increase the transparency of the patient's skin, resulting in improvements in optical imaging measurements.

Tartrazine, also known as FD&C Yellow 5, E number E102, C.I. 19140, FD&C Yellow 5, Yellow 5 Lake, Acid Yellow 23, Food Yellow 4, and trisodium 1-(4-sulfonatophenyl)-4-(4-sulfonatophenylazo)-5-pyrazolone-3-carboxylate, is one such dye that has been shown to increase the transparency of skin, when applied to the outer surface of the skin. The application of the dye to a patient skin prior at a location A, followed by taking an optical measurement at that same location A, may increase the accuracy and ease of data collection in relation to blood oxygen levels, for example using pulse oximetry.

In some forms of the present technology, a fluid transfer apparatus 3900

has been developed for use with an RPT system, the fluid transfer apparatus providing a reservoir for storing a product, and a delivery system for transferring the product from the reservoir to the skin of a patient. The fluid transfer apparatus of the present technology may be provided as a separate component to be used with the RPT system, or may be incorporated into existing components such as the patient interface, for ease of use when the patient interface is being used by the wearer.

FIGS. 6-8 show some examples of the fluid transfer apparatus 3900 of the present technology.

FIGS. 6, 6A and 6B each show components of a forehead support 3700, forehead support 3700 including a forehead frame 3720 configured to define apertures 3721 for receiving an additional member to the forehead frame. In the examples shown, a backing plate 3710 is configured with attachment lugs 3712, attachment lugs 3712 configured to be received within apertures 3721 of forehead frame 3720. Backing plate 3710 is shaped to provide a surface to either comfortably abut a patient's forehead, and may include two wing regions that sit either side of the centre of the patient's forehead, spreading the force evenly between both sides.

In some forms of the technology, a comfort pad 3730 may be configured to removably connected to a face of the backing plate 3710 opposing the forehead frame 3720, the comfort pad 3730 formed from a material the creates a softer surface to place against the patient's forehead, and/or to provide added functionality to the patient interface.

FIGS. 6C and 6D show a simplified diagram of a fluid transfer apparatus 3900 of the present technology. FIG. 6C shows a fluid transfer apparatus 3900 comprising at least one reservoir 3910 for containing a product to be provided to a patient, and at least one fluid delivery system 3920 fluidly connected to the reservoir 3910, the fluid delivery system configured to be able to provide the product to a patient.

FIG. 6D exemplifies a further form of the technology where the fluid transfer apparatus 3900 further comprises an optical sensor 3930 to enable optical imaging and a transmitter 3940, or optical signal transmitter for providing information received by the sensor to a processor or RPT component for analysis.

In some forms, the reservoir may be at least one chamber, or may be multiple chambers that may or may not be fluidly connected together. Multiple chambers within a reservoir would enable the transfer of more than one type of product, with the products able to be kept separate from each other prior to delivery. This may be different types of dye, or skin treatment products that may be applied together or sequentially, different concentrations of dyes, dye additives that may optimise skin transparency further for optical imaging, fluorescent tracers, or it may be that the different chambers enable products with different states to be held apart, before being mixed together for application. In one example this may be a reservoir that holds a dye powder in one chamber and a solvent in a second chamber, the two products mixing and being applied to the patient's skin using the fluid delivery system 3920.

Fluid delivery system 3920 is configured to enable movement of a fluid from reservoir 3910 to application on a patient's skin surface that abuts at least a portion of the fluid transfer apparatus 3900.

In some forms, the reservoir may contain multiple chambers fluidly connected in sequence, so a product may be combined with another before delivery to a patient. The reservoir may be configured to be reusable and refillable, or may be a single-use reservoir that is replaced once the contents of the reservoir has been used.

In some forms of the invention and as seen in FIG. 6A, comfort pad 3730 may be configured to comprise a fluid transfer apparatus 3900. In some forms, designed for use with a forehead support, the comfort pad 3730 is shaped to conform to the shape of the backing plate 3710 and has incorporated on and/or in the comfort pad a fluid delivery system 3920 in the form of an absorbent pad, a reservoir 3710 for storing a product to be delivered to the forehead of the patient, the fluid delivery system 3920 configured to enable product (for example a dye) to be absorbed from the reservoir 3710 onto a first surface of the absorbent pad and transferred through the absorbent pad to an opposing second surface of the absorbent pad, such that the product is then provided to the forehead of a patient, when the forehead support with comfort pad if in place on the user's forehead.

The absorbent pad may have a range of different thickness, with a greater thickness typically enabling storage of a greater amount of product. The thickness of the absorbent pad may be modified to enable control of how much of the product is provided to the patient, and the pad may be made with materials or a combination of materials having different absorbencies, so as to control the rate at which the product is transferred from the reservoir to the skin, and/or the amount of product. The absorbent materials may be chosen to enable movement of a fluid in a single direction through the fluid delivery system.

In some instances, it may be desirable to have the product applied in a single instance to reduce skin transparency for a single optical imaging reading, or it may be desirable to have the product applied in amounts over a long period of time, to assist in taking multiple measurements through the skin over a time period.

Sensor 3930 may be incorporated into or onto the comfort pad at one or more locations at or near the fluid delivery system 3920. In use, the fluid delivery system provides a product to the skin that increases the transparency of the skin in the area where the product is applied. This enables a reading taken by a sensor at that same location to be more accurate. As such, when incorporated into the fluid transfer apparatus, a sensor will be located close to the fluid delivery system in order take any required readings at the same location on the patient's skin as the product has been applied. In the example shown in FIG. 6A, the comfort pad includes two wings, with two sensors, one sensor 3930 centrally located on each wing and surrounded or partially surrounded by a reservoir 3710. Each reservoir 3710 is surrounded or partially surrounded by a fluid delivery system 3920. This configuration allows the three components to operate in close proximity to each other ensuring the skin preparation and measurement can happen effectively in the same location.

In other forms not shown, the different components of the fluid transfer apparatus may be configured at or near each other in a variety of orientations that suit the component they are to be incorporated with, or the skin region they are designed to be acting on.

FIGS. 7, 7A and 7B show one form of the invention where fluid transfer apparatus 3900 is mounted on one or more tubes 3350 of a patient interface 3000, and positioned such that fluid transfer apparatus will align with or near the temple (or both temples if mounted on either side of the patient interface 3000) of a patient when in position on the patient's head, such that in use, the fluid delivery system will supply a dye or similar product to the skin area around the temple, and a measurement of blood oxygen concentration (or other measurement taken using optical imaging) may be taken at this site.

FIGS. 8 and 8A show a further embodiment where the fluid transfer apparatus is incorporated within or formed as a circumaural or super-aural earpiece 3960, surrounding or partially surrounding the ear of a patient. This apparatus may be used independently of a RPT system or may be connected or connectable to a patient interface. In the over-ear embodiments, the earpiece 3960 may comprise a head section 3961 that abuts at or near a patient's temple when worn, a hook section 3962 that extends over the top of the ear and a tail section 3693 that extends along the superior side of the ear. In some forms, the reservoir, fluid delivery system and other optional components may be located in the head section 3961, in the hook region, or in the tail section 3963. The location of the components will be configured to ensure they are likely to locate against a region of skin that is preferably hair-free, or has minimal coarse hair, such as the hair on a person's head. While the apparatus may still be useful in a region of more hair, lesser amounts of hair may result in greater skin transparency and potentially more accurate sensor readings.

Tail section 3963 may be varied in length, to effectively house the required components, or to enable the placement of the fluid delivery system at different locations on the head, face or neck.

FIG. 9 shows a further form of the technology, where fluid transfer apparatus 3900 is configured as a finger mounted device. A finger mounted apparatus 3900 is configured such that the apparatus may have an interfacing surface 3902 configured be secured against a pad, for example a distal finger pad, of a patient's finger and secured in position using connecting straps 3901. The fluid delivery system 3920 may be positioned within the apparatus 3900 such that fluid is delivered to the pad of the finger, increasing the transparency of the skin at this location. Reservoir 3910 may be held in, or form the body of the fluid transfer apparatus, and configured to directly to indirectly supply the fluid delivery system with the product to be transferred.

An optical sensor 3930 may be incorporated with the fluid delivery apparatus 3900 and be presented on the interfacing surface 3902 of the apparatus 3900, such that when the apparatus 3900 is in position on a finger, the sensor may take a reading through the user's skin at the location of dye application.

The fluid transfer apparatus of the present technology may include a range of components to allow for the receipt, storage and/or transmission of information collected using an optical sensor 3930.

The sensor may be an optical sensor, for example a sensor configured for pulse-oximetry, near-infrared spectroscopy, diffuse-optical tomography, and/or photoacoustic imaging. The sensor may be configured to receive optical signals or light over the electromagnetic spectrum.

The fluid transfer apparatus may comprise a light source 3945 to enhance or enable optical imaging. The light source 3945 may be positioned on a surface configured to interface with a patient's skin, and may be a light source 3945 such as a single or multi frequency LED, or may be incorporated within the product being transferred to the patient's skin, for example as a fluorescent tracer, or fluorescent element.

In some examples the light source may be separate from, or incorporated within the optical sensor.

In some forms, the fluid transfer apparatus 3900 may have one or more of an electrical power supply 3981, one or more input devices, a controller, a processor/microprocessor 3982, memory, one or more transducers, a wired or wireless transmitter, a data communication interface 3983 and one or more output devices. Electrical components may be mounted on a single Printed Circuit Board Assembly (PCBA).

The fluid transfer apparatus 3900 may be configured to collect and store data on a storage medium 3990 relating to a patient's health and may be configured to transmit the information collected to a RPT system. The RPT system may be configured to respond to the information provided, by altering the output of the therapy to the patient.

The fluid transfer apparatus 3900 may be used as a component in a method or system for the diagnosis, screening and/or treatment of obstructive sleep apnea (OSA) in subjects. 4.5 METHODS OF DIAGNOSIS, SCREENING AND TREATMENT OF OSA

The application of a diagnostic dye that improves the translucence of a subject's skin allows for improvements in optical imaging techniques that rely on conducting measurements through the skin of a subject. The diagnosis of OSA in a subject is often determined using measurements of, or changes in blood volume or blood oxygen concentration for example, using techniques such as for example, photoplethysmography.

By improving the accuracy, speed or ease of measurement of such analysis, diagnosis of OSA may be improved, as well as monitoring of subjects over time to screen for the onset of, or improvements in OSA. The fluid transfer apparatus above discloses a technology whereby a a diagnostic dye may be incorporated into an existing respiratory therapy system to provide a dye to the skin that results in the formation of a measurement site on the subject's skin that has an increased transparency.

FIGS. 10-11 show an overview of the methods of this technology.

Method 105 of FIG. 10 shows a method for the diagnosis or screening of OSA in some forms of the technology.

In performing the method, a diagnostic dye is administered to 105a to an area of a subject's skin where a measurement is to be taken. This area is preferably free of or limited in the amount of hair growth to ensure an accurate reading. In some examples this may be a subject's face, neck, hand/fingers, arm, chest or torso, or any other region where clear skin contact may be made.

A preferred diagnostic dye is tartrazine, and this may be applied once, or multiple times over a period of time to increase the translucence of skin at the area of application. Depending on the skin tone of the patient, the interaction time may increase or decrease to achieve the desired transparency, or different regions of the skin may be targeted to achieve the best results based on skin tone at a particular area.

Any improvement in the translucence of skin compared to standard skin translucency is an advantage. The area to which the diagnostic dye is applied may be altered to suit the type of optical sensor being used for measurement at the site, but in some examples, the area may range from 5 mm2-50 mm2.

Following application of the dye 105a, light is provided to the surface of the skin 105b to generate optical information that is to be received by an optical sensor. The light source may be incorporated into a fluid transfer apparatus, or optical sensor, or may be a standalone light source. Such a light source may be a single or multi-frequency LED, laser, halogen lamps or arc-lamps for example and may be selected based on the wave-length of light required.

When using photoplethysmography as a measurement technique, light from the light source is provided to the subject's skin, and the light is scattered or absorbed over time, with the variance depending on each heartbeat. This light is then reflected or transmitted back to the optical sensor, in this example a photodetector, where the changes in the light are measured 105c.

The changes in the measured light are analyzed 105d to determine parameters such as blood volume, heart rate, respiratory rate or blood oxygenation and measurements may be taken as a single reading, or various reading may be taken over time to identify changes over the time period. In one example this may involve taken multiple readings over period of sleep to monitor changes on blood oxygen, heart rate or blood volume over time. This may then be used to identify measurements that correlate with known or expected measurements 105e associated with the presence of sleep apena.

Determination of a positive or negative diagnosis of sleep apnea involves a comparison of measured data against expected measurements or ranges of measurements. A positive OSA indicator may be found if one or more measurement values fall outside an predetermined range that in indicative of a healthy individual without sleep apnea. In one example, a healthy individual may have an average blood oxygenation of 95%-100% over a period of sleep. If measurements are taken with the current method using photoplethysmography and a subject's average blood oxygen levels are below 95%, this may trigger a positive diagnosis of sleep apnea, or an indication that further analysis may be required. Other measurements that may be used for comparison include but are not limited to blood volume or blood volume changes over time, heart rate, respiratory rate, blood oxygenation or blood CO2 levels.

Different criteria may be used to compare and determine a positive diagnosis of sleep apnea, such as individual readings outside a normal range, or a certain number of outlying measurements over time. Equally, measurements that fall within the expected ranges or values of a healthy subject without sleep apnea (for example 98% oxygenation over a time period) may result in a negative diagnosis of sleep apnea being provided.

Once a positive of negative determination of OSA is completed, a positive or negative diagnosis is provided to the patient.

As seen in FIG. 11, in some case, the optical imaging measurements may be extrapolated to determine a phenotypic cause of OSA. Such phenotypic causes that may be indicated by the optical imaging measurements may include, but are not limited to passive critical closing pressure of the upper airway (Pcrit), genioglossus muscle responsiveness, arousal threshold, and/or respiratory control stability.

Treatment of OSA may be conducted using known techniques such as respiratory therapy, using the respiratory therapy apparatus and systems described herein. In other cases, therapy techniques may include positive airway pressure (PAP), mandibular adjustment devices, or pharmacological treatments.

The application of the diagnostic dye allows the method of diagnosing to be accurately and effectively undertaken as a result of the improved optical measurement through the skin tissue. The increased transparency provides more accurate readings using the optical imaging techniques, which results in more accurate diagnosis and potentially the recommendation for more effective treatments, improving overall patient outcomes.

### 4.6 Systems for Diagnosis, Screening and Treatment of OSA

The methods of screening and diagnosis of the present invention may be implemented using a system for the diagnosis and screening of OSA using optical sensing in conjunction with improved skin translucency. The system may be used as a standalone system, or may be used in conjunction with a respiratory therapy system for ongoing monitoring of a subject's health and symptoms of OSA, as well as providing information regarding the efficacy of an OSA treatment protocol.

FIG. 12 shows a system 6000 for the diagnosis and screening of OSA in certain forms of the technology utilising a fluid transfer apparatus as described in further detail above. In other forms, other means of applying fluid to a subject's skin tissue may be utilised, such as self-application using swabs or absorbent pads for example.

System 6000 further comprises a light source configured to provide light to the subject's skin tissue at the measurement site. The light source may be incorporated into a fluid transfer apparatus, or optical sensor, or may be a standalone light source. Such a light source may be a single or multi-frequency LED, laser, halogen lamps or arc-lamps for example and may be selected based on the wave-length of light required. The light source may be directed or contained in an apparatus that ensures the required direction and focussing of the light source to the skin tissue as required.

When using photoplethysmography as a measurement technique (as an example), light from the light source is provided to the subject's skin, and the light is scattered or absorbed over time, with the variance depending on each heartbeat. This light is then reflected or transmitted back to the optical sensor, in this example a photodetector, where the changes in the light are measured. Measurements may be taken as a single reading, or various reading may be taken over time to identify changes over the time period. In one example this may involve taken multiple readings over period of sleep to monitor changes on blood oxygen, heart rate or blood volume over time.

The system may further comprise a transmitter 3940 to transmit information from the optical sensor 3930 to a processor 3982, where changes in the measured light are analyzed to determine parameters such as blood volume, heart rate, respiratory rate or blood oxygenation. In some embodiments, the processor may be incorporated within the optical sensor and a separate transmission function is not required.

Processor 3982 may be used to analyse the measured parameters and compare them with predetermined parameters associated with the presence of sleep apena. For example, determination of a positive or negative diagnosis of sleep apnea involves a comparison of measured data against expected measurements or ranges of measurements. A positive OSA indicator may be found if one or more measurement values fall outside an predetermined range that in indicative of a healthy individual without sleep apnea. In one example, a healthy individual may have an average blood oxygenation of 95%-100% over a period of sleep. If measurements are taken with the current method using photoplethysmography and a subject's average blood oxygen levels are below 95%, this may trigger a positive diagnosis of sleep apnea, or an indication that further analysis may be required. Other measurements that may be used for comparison include but are not limited to blood volume or blood volume changes over time, heart rate, respiratory rate, blood oxygenation or blood CO2 levels.

Different criteria may be used to compare and determine a positive diagnosis of sleep apnea, such as individual readings outside a normal range, or a certain number of outlying measurements over time. Equally, measurements that fall within the expected ranges or values of a healthy subject without sleep apnea (for example 98% oxygenation over a time period) may result in a negative diagnosis of sleep apnea being provided.

System 6000 may further comprise an interface 3983 configured to provide information about the OSA diagnosis to a user or external device. The interface may be a graphical user interface, or may be an interface that communicates with an external device to provide information from the optical sensor to the user.

The system 6000 may in some forms comprise a power source and/or a data storage medium.

In certain embodiments, the system may also comprise includes known OSA treatment methods, such as positive airway pressure (PAP) using a respiratory therapy device such as the one described herein (for example only), mandibular adjustment devices, or pharmacological treatments. These treatment methods are integrated into the system to provide a comprehensive approach to managing OSA. The system is configured to accurately determine the best cause of treatment for the subject using these known treatment methods. This determination is based on the analysis of optical signals and/or the phenotypic causes of OSA.

These phenotypic causes may include passive critical closing pressure of the upper airway [Pcrit], genioglossus muscle responsiveness, arousal threshold, and respiratory control stability. By understanding these phenotypic causes, the system can provide targeted treatment recommendations that are tailored to the individual needs of the subject.

Additionally, the system is designed to enhance diagnostic and screening capabilities. This enhancement may be achieved through the use of machine learning or AI, which can analyze large datasets and provide more accurate diagnostic and treatment recommendations.

Overall, the OSA diagnostic and screening system 6000, along with its sub-components, provides a comprehensive and advanced approach to diagnosing and treating OSA. By utilizing optical diagnostics and screeners, known treatment methods, and advanced technologies such as machine learning and AI, the system can accurately diagnose OSA, determine the best cause of treatment, and enhance overall diagnostic and screening capabilities.

### 4.7 RPT Device

An RPT device 4000 in accordance with one aspect of the present technology comprises mechanical, pneumatic, and/or electrical components and is configured to execute one or more algorithms 4300, such as any of the methods, in whole or in part, described herein. The RPT device 4000 may be configured to generate a flow of air for delivery to a patient's airways, such as to treat one or more of the respiratory conditions described elsewhere in the present document.

### 4.7.1 RPT Device Algorithms

As mentioned above, in some forms of the present technology, the central controller 4230 may be configured to implement one or more algorithms 4300 expressed as computer programs stored in a non-transitory computer readable storage medium, such as memory 4260. The algorithms 4300 are generally grouped into groups referred to as modules.

In other forms of the present technology, some portion or all of the algorithms 4300 may be implemented by a controller of an external device such as the local external device 4288 or the remote external device 4286. In such forms, data representing the input signals and/or intermediate algorithm outputs necessary for the portion of the algorithms 4300 to be executed at the external device may be communicated to the external device via the local external communication network 4284 or the remote external communication network 4282. In such forms, the portion of the algorithms 4300 to be executed at the external device may be expressed as computer programs, such as with processor control instructions to be executed by one or more processor(s), stored in a non-transitory computer readable storage medium accessible to the controller of the external device. Such programs configure the controller of the external device to execute the portion of the algorithms 4300.

In such forms, the therapy parameters generated by the external device via the therapy engine module 4320 (if such forms part of the portion of the algorithms 4300 executed by the external device) may be communicated to the central controller 4230 to be passed to the therapy control module 4330.

### 4.7.2 Air Circuit

An air circuit 4170 in accordance with an aspect of the present technology is a conduit or a tube constructed and arranged to allow, in use, a flow of air to travel between two components such as RPT device 4000 and the patient interface 3000 or 3800.

In particular, the air circuit 4170 may be in fluid connection with the outlet of the pneumatic block 4020 and the patient interface. The air circuit may be referred to as an air delivery tube. In some cases, there may be separate limbs of the circuit for inhalation and exhalation. In other cases, a single limb is used.

In some forms, the air circuit 4170 may comprise one or more heating elements configured to heat air in the air circuit, for example to maintain or raise the temperature of the air. The heating element may be in a form of a heated wire circuit, and may comprise one or more transducers, such as temperature sensors. In one form, the heated wire circuit may be helically wound around the axis of the air circuit 4170. The heating element may be in communication with a controller such as a central controller 4230. One example of an air circuit 4170 comprising a heated wire circuit is described in U.S. Pat. No. 8,733,349, which is incorporated herewithin in its entirety by reference.

### 4.7.3 Supplementary Gas Delivery

In one form of the present technology, supplementary gas, e.g. oxygen, 4180 is delivered to one or more points in the pneumatic path, such as upstream of the pneumatic block 4020, to the air circuit 4170, and/or to the patient interface 3000 or 3800.

### 4.7.4 Non-Obtrusive Monitoring System

One example of a monitoring apparatus 7000 for monitoring the respiration of a sleeping patient 1000 contains a contactless motion sensor generally directed toward the patient 1000 and may further comprise a fluid transfer apparatus. The motion sensor is configured to generate one or more signals representing bodily movement of the patient 1000, from which may be obtained a signal representing respiratory movement of the patient. The fluid transfer apparatus may optimise the collection of oxygen saturation data from a pulse oximeter.

### 4.7.5 Respiratory Polygraphy

Respiratory polygraphy (RPG) is a term for a simplified form of PSG without the electrical signals (EOG, EEG, EMG), snore, or body position sensors. RPG comprises at least a thoracic movement signal from a respiratory inductance plethysmogram (movement sensor) on a chest band, e.g. the movement sensor 2040, a nasal pressure signal sensed via a nasal cannula, and an oxygen saturation signal from a pulse oximeter, e.g. the pulse oximeter 2055. The three RPG signals, or channels, are received by an RPG headbox, similar to the PSG headbox 2000.

In certain configurations, a nasal pressure signal is a satisfactory proxy for a nasal flow rate signal generated by a flow rate transducer in-line with a sealed nasal mask, in that the nasal pressure signal is comparable in shape to the nasal flow rate signal. The nasal flow rate in turn is equal to the respiratory flow rate if the patient's mouth is kept closed, i.e. in the absence of mouth leaks.

FIG. 9 is a block diagram illustrating a screening/diagnosis/monitoring device 7200 that may be used to implement an RPG headbox in an RPG screening/diagnosis/monitoring system. The screening/diagnosis/monitoring device 7200 receives the three RPG channels mentioned above (a signal indicative of thoracic movement, a signal indicative of nasal flow rate, and a signal indicative of oxygen saturation) at a data input interface 7260. The screening/diagnosis/monitoring device 7200 also contains a processor 7210 configured to carry out encoded instructions. The screening/diagnosis/monitoring device 7200 also contains a non-transitory computer readable memory/storage medium 7230.

Memory 7230 may be the screening/diagnosis/monitoring device 7200's internal memory, such as RAM, flash memory or ROM. In some implementations, memory 7230 may also be a removable or external memory linked to screening/diagnosis/monitoring device 7200, such as an SD card, server, USB flash drive or optical disc, for example. In other implementations, memory 7230 can be a combination of external and internal memory. Memory 7230 includes stored data 7240 and processor control instructions (code) 7250 adapted to configure the processor 7210 to perform certain tasks. Stored data 7240 can include RPG channel data received by data input interface 7260, and other data that is provided as a component part of an application. Processor control instructions 7250 can also be provided as a component part of an application program. The processor 7210 is configured to read the code 7250 from the memory 7230 and execute the encoded instructions. In particular, the code 7250 may contain instructions adapted to configure the processor 7210 to carry out methods of processing the RPG channel data provided by the interface 7260. One such method may be to store the RPG channel data as data 7240 in the memory 7230. Another such method may be to analyse the stored RPG data to extract features. The processor 7210 may store the results of such analysis as data 7240 in the memory 7230.

The screening/diagnosis/monitoring device 7200 may also contain a communication interface 7220. The code 7250 may contain instructions configured to allow the processor 7210 to communicate with an external computing device (not shown) via the communication interface 7220. The mode of communication may be wired or wireless. In one such implementation, the processor 7210 may transmit the stored RPG channel data from the data 7240 to the remote computing device. In such an implementation, the remote computing device may be configured to analyse the received RPG data to extract features. In another such implementation, the processor 7210 may transmit the analysis results from the data 7240 to the remote computing device.

Alternatively, if the memory 7230 is removable from the screening/diagnosis/monitoring device 7200, the remote computing device may be configured to be connected to the removable memory 7230. In such an implementation, the remote computing device may be configured to analyse the RPG data retrieved from the removable memory 7230 to extract the features.

### 4.8 GLOSSARY

For the purposes of the present technology disclosure, in certain forms of the present technology, one or more of the following definitions may apply. In other forms of the present technology, alternative definitions may apply.

### 4.8.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’ is sometimes shortened to simply ‘flow’ or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar 0.001 atm). In this specification, unless otherwise stated, pressure is given in units of cmH2O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 4.9 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Furthermore, “approximately”, “substantially”, “about”, or any similar term used herein means +/−5-10% of the recited value.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

### 4.10 REFERENCE SIGNS LIST

| | |
|---|---|
| patient | 1000 |
| bed partner | 1100 |
| headbox | 2000 |
| ground electrode ISOG | 2010 |
| EOG electrode | 2015 |
| EEG electrode | 2020 |
| ECG electrode | 2025 |
| submental EMG electrode | 2030 |
| snore sensor | 2035 |
| respiratory inductance plethysmogram | 2040 |
| respiratory inductance plethysmogram | 2045 |
| respiratory effort sensor | |
| oro - nasal cannula | 2050 |
| pulse oximeter | 2055 |
| body position sensor | 2060 |
| patient interface | 3000 |
| nose and mouth patient interface | 3000-1 |
| nose and mouth patient interface | 3000-2 |
| nose-only patient interface | 3000-3 |
| nose-only patient interface | 3000-4 |
| mouth cushion | 3050-1 |
| nose cushion | 3050-2 |
| seal - forming structure | 3100 |
| cushion module | 3150 |
| plenum chamber | 3200 |
| plenum chamber | 3200-1 |
| plenum chamber | 3200-2 |
| chord | 3210 |
| superior point | 3220 |
| inferior point | 3230 |
| plenum chamber inlet port | 3254 |
| plenum chamber inlet port | 3254-1 |
| plenum chamber inlet port | 3254-2 |
| groove | 3266-1 |
| groove | 3266-2 |
| positioning and stabilising structure | 3300 |
| headgear | 3302 |
| headgear | 3302-1 |
| headgear | 3302-2 |
| inferior strap | 3304-1 |
| inferior strap | 3304-2 |
| superior strap | 3305-1 |
| magnetic member | 3306-1 |
| rear strap | 3307-1 |
| rear strap | 3307-2 |
| intermediate section | 3308-2 |
| slit | 3309-2 |
| strap | 3310 |
| tab | 3320 |
| concertina structure | 3328 |
| inlet | 3332 |
| rigidiser arm | 3340 |
| first end | 3342 |
| second end | 3344 |
| tube | 3350 |
| sleeve | 3351 |
| superior opening | 3352 |
| inferior extension | 3354 |
| connection member | 3356 |
| non - extendable tube sections | 3363 |
| magnet | 3370-1 |
| four - point arm sleeve | 3380 |
| conduit sleeve | 3380-1 |
| inferior extension | 3384 |
| connection member | 3386 |
| inferior opening | 3388-1 |
| tabs | 3394 |
| tabs | 3394-1 |
| vent | 3400 |
| vent opening | 3402 |
| vent opening | 3402-1 |
| vent opening | 3402-2 |
| vent housing | 3404 |
| anterior surface | 3408 |
| posterior surface | 3412 |
| groove | 3416 |
| diffuser | 3448 |
| vent | 3450 |
| conduit connection structure | 3500 |
| arm connection structure | 3504 |
| connection port | 3600 |
| elbow | 3610 |
| forehead support | 3700 |
| ISO | 3744 |
| unsealed patient interface | 3800 |
| prongs | 3810a |
| prongs | 3810b |
| lumens | 3820a |
| lumens | 3820b |
| fluid transfer apparatus | 3900 |
| optical sensor | 3930 |
| transmitter | 3940 |
| light source | 3945 |
| processor | 3982 |
| interface | 3983 |
| RPT device | 4000 |
| external housing | 4010 |
| upper portion | 4012 |
| portion | 4014 |
| panel | 4015 |
| chassis | 4016 |
| handle | 4018 |
| pneumatic block | 4020 |
| air filter | 4110 |
| inlet air filter | 4112 |
| outlet air filter | 4114 |
| muffler | 4120 |
| inlet muffler | 4122 |
| outlet muffler | 4124 |
| pressure generator | 4140 |
| blower | 4142 |
| motor | 4144 |
| anti - spill back valve | 4160 |
| air circuit | 4170 |
| air circuit | 4171 |
| supplementary gas | 4180 |
| electrical components | 4200 |
| single Printed Circuit Board Assembly | 4202 |
| PCBA | |
| power supply | 4210 |
| input device | 4220 |
| central controller | 4230 |
| clock | 4232 |
| therapy device controller | 4240 |
| protection circuits | 4250 |
| memory | 4260 |
| transducer | 4270 |
| pressure sensor | 4272 |
| flow rate sensor | 4274 |
| motor speed transducer | 4276 |
| data communication interface | 4280 |
| remote external communication network | 4282 |
| local external communication network | 4284 |
| remote external device | 4286 |
| local external device | 4288 |
| output device | 4290 |
| display driver | 4292 |
| display | 4294 |
| algorithms | 4300 |
| pre - processing module | 4310 |
| interface pressure algorithm | 4312 |
| vent flow rate estimation algorithm | 4314 |
| leak flow rate estimation algorithm | 4316 |
| respiratory flow rate estimation | 4318 |
| algorithm | |
| therapy engine module | 4320 |
| phase determination algorithm | 4321 |
| waveform determination | 4322 |
| ventilation determination | 4323 |
| inspiratory flow limitation determination | 4324 |
| apnea / hypopnea determination | 4325 |
| snore determination | 4326 |
| airway patency determination algorithm | 4327 |
| target ventilation determination | 4328 |
| algorithm | |
| therapy parameter determination | 4329 |
| algorithm | |
| therapy control module | 4330 |
| corresponding algorithm | 4340 |
| method | 4500 |
| step | 4520 |
| step | 4530 |
| step | 4540 |
| step | 4550 |
| step | 4560 |
| humidifier | 5000 |
| humidifier inlet | 5002 |
| humidifier outlet | 5004 |
| humidifier base | 5006 |
| reservoir | 5110 |
| conductive portion | 5120 |
| humidifier reservoir dock | 5130 |
| locking lever | 5135 |
| water level indicator | 5150 |
| humidifier transducer | 5210 |
| air pressure sensor | 5212 |
| air flow rate transducer | 5214 |
| temperature sensor | 5216 |
| humidity sensor | 5218 |
| heating element | 5240 |
| humidifier controller | 5250 |
| central humidifier controller | 5251 |
| heating element controller | 5252 |
| air circuit controller | 5254 |
| system for diagnosis, screening and | |
| treatment of OSA | |
| screening / diagnosis / monitoring | 7200 |
| device | |
| processor | 7210 |
| communication interface | 7220 |
| memory | 7230 |
| data | 7240 |
| code | 7250 |
| data input interface | 7260 |
| interface | 7260 |

1. A fluid transfer apparatus comprising:
at least one reservoir for containing at least one product; and
at least one fluid delivery system fluidly connectable to the reservoir and comprising an application member having a patient contacting surface;
the fluid delivery system configured such that in use, the fluid delivery system may receive at least one product from the reservoir and provide at least one product in fluid form to the patient contacting surface of the application member.

2. The fluid transfer apparatus of claim 1, wherein the product is a fluid in the form of a dye and/or contrast media.

3. The fluid transfer apparatus of claim 2, wherein the dye is a diagnostic dye suitable for increasing skin and/or soft tissue transparency.

4. The fluid transfer apparatus of claim 3, wherein the diagnostic dye is tartrazine.

5. The fluid transfer apparatus of claim 1, wherein the at least one reservoir is configured to be able to contain a dye powder.

6. The fluid transfer apparatus of claim 1, wherein the fluid transfer apparatus comprises two reservoirs.

7. The fluid transfer apparatus of claim 6, wherein the fluid transfer apparatus is configured to enable mixing of at least one product from each of the two reservoirs prior to providing the at least one product to the patient contacting surface of the application member.

8. The fluid transfer apparatus of claim 1, wherein the fluid transfer apparatus further comprises one or more of: an optical sensor; a light source; a transmitter; a processor; a storage medium; and/or a power source.

9. The fluid transfer apparatus of claim 1, wherein the fluid delivery system comprises an absorbent pad.

10. The fluid transfer apparatus of claim 1, wherein the application member of the fluid transfer apparatus comprises at least one reservoir interface and a patient contacting surface, the at least one reservoir interface configured to enable a fluid connection between the at least one reservoir and the patient contacting surface.

11. The fluid transfer apparatus of claim 10, wherein the at least one reservoir interface is configured to enable a fluid connection between the reservoir and the patient contacting surface to be open, partially open or closed.

12. The fluid transfer apparatus of claim 1, wherein the fluid transfer apparatus is a component in, or for use with a respiratory therapy (RPT) system.

13. The fluid transfer apparatus of claim 12, wherein the fluid transfer apparatus forms part of a patient interface in an RPT system and/or is configured to be removably attached to the patient interface.

14. The fluid transfer apparatus of claim 13, wherein the patient interface comprises a forehead support, and the fluid transfer apparatus is configured to form part of the forehead support, and/or configured to be removably attached to the forehead support.

15. The fluid transfer apparatus of claim 13, wherein the fluid transfer apparatus forms part of the positioning and stabilising device, and/or is configured to be removably attached to the positioning and stabilising device.

16. The fluid transfer apparatus of claim 12, wherein the fluid transfer apparatus is configured as a finger mounted module.

17. A patient interface for use with a respiratory therapy (RPT) system, the patient interface comprising the fluid transfer apparatus of claim 1, the fluid transfer apparatus configured with the patient interface to facilitate connection between the fluid transfer apparatus and a region of skin of the patient when the patient interface is in use.

18. A method for diagnosing and screening obstructive sleep apnea (OSA) in a subject, the method comprising;
a) administering a diagnostic dye to the subject's skin to increase skin transparency at a measurement site using the fluid transfer apparatus of claim 1;
b) providing light to the subject's skin tissue at the measurement site using a light source;
c) receiving reflected or transmitted light from the measurement site using an optical sensor;
d) analysing the reflected or transmitted light received from the measurement site to determine at least one measured parameter;
e) comparing the measured parameter to at least one predetermined threshold or at least one range of parameters associated with an indication of OSA;
f) determining if the at least one measured parameter indicates the patient is positive for OSA or negative for OSA, based on a comparison of the at least one measured parameter with the at least one predetermined parameter; and
g) providing a positive or negative diagnosis of OSA based on the determination of step f).

19. The method of claim 18, wherein the method further comprises repeating at least steps b)-d) multiple times over a specified period of time and identifying any change(s) in the at least one measured parameter over the specified period of time.

20. The method of claim 18, wherein the method comprises the further step of treating obstructive sleep apnea (OSA) in a subject positively diagnosed with OSA.

21. The method of claim 20, wherein the step of treating OSA comprises at least one of positive airway pressure (PAP), mandibular adjustment devices, or pharmacological treatments.

22. A system for the diagnosis or screening of obstructive sleep apnea (OSA) in a subject, the system comprising;
a) a fluid transfer apparatus of claim 1 to enable increased skin transparency at a measurement site located on the subject;
b) a light source to provide light to the subject's skin tissue at the measurement site;
c) an optical sensor to receive reflected or transmitted light from the measurement site;
d) a transmitter configured to transmit information regarding the reflected or transmitted light from the optical sensor to a processor;
e) a processor configured to analyse the reflected or transmitted light received from the measurement site to determine at least one measured parameter and compare the at least one measured parameter to at least one predetermined threshold or at least one range of parameters associated with an indication of OSA to provide an OSA diagnosis; and
f) an interface to provide information about the OSA diagnosis to a user or external device.

23. The system of claim 22, wherein the optical sensor is configured for pulse-oximetry, near-infrared spectroscopy, diffuse-optical tomography, and/or photoacoustic imaging.

24. The system of claim 22, wherein the light source is a single or multi-frequency LED.

25. The system of claim 22, wherein the system is a system for diagnosis, screening and treatment of obstructive sleep apnea (OSA) and the system further comprises at least one of positive airway pressure (PAP) respiratory therapy system or a mandibular adjustment device.
